(19) United States
(12) Patent Application Publication
(10) Pub. No.: US 2022/0049228 A1
(43) Pub. Date: Feb. 17, 2022

(54) Modified Extracellular Enveloped Virus

(71) Applicant: KaliVir Immunotherapeutics, Inc., Pittsburgh, PA (US)

(72) Inventors: Stephen H. THORNE, Pittsburgh, PA (US); Daniel J. BYRD, Pittsburgh, PA (US); Mingrui ZHANG, Pittsburgh, PA (US)

(21) Appl. No.: 17/494,322

(22) Filed: Oct. 5, 2021

Related U.S. Application Data
(63) Continuation of application No. PCT/US2020/056107, filed on Oct. 16, 2020.
(60) Provisional application No. 62/916,035, filed on Oct. 16, 2019.

(51) Int. Cl. C12N 7/00; A61K 35/768; A61K 45/06

(52) U.S. Cl. CPC C12N 7/00; A61K 35/768; C12N 2710/24132; C12N 2710/24121; C12N 2710/24122; A61K 45/06

(57) ABSTRACT

This disclosure provides a modified oncolytic poxvirus, such as a vaccinia virus, that can contain modifications in the viral genome that increases production of an extracellular enveloped form of the virus. The modified oncolytic poxvirus can be utilized as a vector for systemic delivery. Also provided are methods of using the modified oncolytic poxvirus.

### CROSS-REFERENCE

This application claims the benefit of U.S. Provisional Application No. 62/916,035 filed Oct. 16, 2019, which is incorporated by reference herein in its entirety.

### INCORPORATION BY REFERENCE

All publications, patents, patent applications, and NCBI accession numbers mentioned in this specification are herein incorporated by reference to the same extent as if each individual publication, patent, or patent application was specifically and individually indicated to be incorporated by reference, and as if set forth in their entireties. In the event of a conflict between a term as used herein and the term as defined in the incorporated reference, the definition of this disclosure controls.

### SUMMARY

One embodiment provides a modified oncolytic poxvirus comprising a nucleic acid that codes for an A34R protein or a fragment thereof comprising at least two mutations, wherein the at least two mutations are in positions corresponding to positions Lys119 and Lys151 of a wild-type vaccinia virus A43R protein (SEQ ID NO. 4). In some embodiments, the mutation in the position corresponding to position Lys119 is Lys119Glu. In some embodiments, the mutation in the position corresponding to position Lys151 is Lys151Glu. In some embodiments, the at least two mutations in positions corresponding to positions Lys119 and Lys151 of the wild-type vaccinia virus A43R protein (SEQ ID NO. 4) are Lys119Glu and Lys151Glu, respectively.

One embodiment provides a modified oncolytic poxvirus comprising a nucleic acid that codes for an A34R protein or a fragment thereof comprising at least two non-naturally occurring mutations that are in amino acid residues that are positively charged at pH 5, within the wild-type A34R protein (SEQ ID No. 4).

Another embodiment provides a modified oncolytic poxvirus comprising a nucleic acid that codes for an A34R protein or a fragment thereof comprising at least two non-naturally occurring mutations that are not at position 110 of the wild-type A34R protein (SEQ ID No. 4).

Another embodiment provides a modified oncolytic poxvirus comprising a nucleic acid that codes for an A34R protein or a fragment thereof comprising at least two non-naturally occurring mutations that are not at aspartic acid residues within the wild-type A34R protein (SEQ ID No. 4).

Another embodiment provides a modified oncolytic poxvirus comprising a nucleic acid that codes for an A34R protein or a fragment thereof comprising at least two non-naturally occurring mutations that are independently in alanine, arginine, asparagine, cysteine, glutamine, glutamic acid, glycine, histidine, isoleucine, leucine, lysine, methionine, phenylalanine, proline, serine, threonine, tryptophan, tyrosine, or valine residues within the wild-type A34R protein (SEQ ID No. 4).

Another embodiment provides a modified oncolytic poxvirus comprising a nucleic acid that codes for an A34R protein or a fragment thereof comprising a non-naturally occurring mutation at a lysine residue that is at a position other than position Lys151 of the wild-type A34R protein (SEQ ID No. 4).

Another embodiment provides a modified oncolytic poxvirus comprising a nucleic acid that codes for an A34R protein or a fragment thereof comprising a non-naturally occurring mutation at position Lys119 of the wild-type A34R protein (SEQ ID No, 4).

Another embodiment provides a modified oncolytic poxvirus comprising a nucleic acid that codes for an A34R protein or a fragment thereof comprising at least two non-naturally occurring mutations that are in amino acid residues that are positively charged at pH 5, within the wild-type A34R protein (SEQ ID No. 4), wherein the modified oncolytic poxvirus generates an increased number of comet tail type plaques in a viral plaque forming assay, compared to an otherwise identical oncolytic virus that does not comprise the at least two non-naturally occurring mutations.

Another embodiment provides a modified oncolytic poxvirus comprising a nucleic acid that codes for an A34R protein or a fragment thereof comprising at least two non-naturally occurring mutations, wherein if either of the non-naturally occurring mutations are at position 110 within the wild-type A3%1R protein (SEQ ID No. 4), the amino acid encoded is not an asparagine residue.

Another embodiment provides a modified oncolytic poxvirus that demonstrates an increased resistance to neutralizing antibodies compared to a wild-type strain of the oncolytic poxvirus, wherein the increased resistance is measured by number of plaques produced by the modified oncolytic poxvirus or the wild-type strain in a viral plaque assay, following treatment with an anti-L1 NR-45114 antibody or an anti-VIG antibody, and wherein the modified oncolytic poxvirus produces at least about 55,000 plaque forming units/mL.

Another embodiment provides a modified oncolytic poxvirus that produces at least about 55,000 plaque forming units/mL, in a viral plaque assay, following treatment with a neutralizing antibody.

In some embodiments, the neutralizing antibody is an anti-L1 NR-45114 antibody or an anti-VIG antibody. In some embodiments, the A34R protein or the fragment thereof further comprises a mutation at position Lys151 of the wild-type A34R protein (SEQ ID No. 4). In some embodiments, the amino acid residues that are positively charged at pH 5 are lysine residues. In some embodiments, the nucleic acid comprises a nucleotide sequence that is at least about 80% homologous to the coding sequence within the viral gene VACWR157, or a fragment thereof. In some embodiments, the nucleic acid comprises a nucleotide sequence that is at least about 80% homologous to the nucleotide sequence set forth as SEQ ID No. 3. In some embodiments, at least one of the two non-naturally occurring mutations is at position Lys119 of the wild-type A34R protein (SEQ ID No. 4). In some embodiments, the non-naturally occurring mutation is at position Lys119 of the wild-type A34R protein (SEQ ID No. 4). In some embodiments, the mutation at position Lys119 of the wild-type A34R protein (SEQ ID No. 4) is Lys119Glu. In some embodiments, at least one of the two non-naturally occurring mutations is at position Lys151 of the wild-type A34R protein (SEQ ID No. 4). In some embodiments, the mutation at position Lys151 of the wild-type A34R protein (SEQ ID No. 4) is Lys151Glu.

One embodiment provides a modified oncolytic poxvirus that expresses an A34R protein comprising mutations Lys119Glu and Lys151Glu.

In some embodiments, positions 305-307 of SEQ ID No. 3 comprises nucleotides GAA or GAG. In some embodiments, positions 451-453 of SEQ ID No. 3 comprises nucleotides GAA or GAG. In some embodiments, the modified oncolytic poxvirus produces a greater amount of an extracellular enveloped virus form than an intracellular mature virus form, as compared to an otherwise identical oncolytic virus that does not comprise the at least two non-naturally occurring mutations. In some embodiments, the modified oncolytic poxvirus produces a greater amount of an extracellular enveloped virus form than an intracellular mature virus form, as compared to an otherwise identical oncolytic virus that does not comprise the non-naturally occurring mutation. In some embodiments, the modified oncolytic poxvirus further comprises an exogenous nucleic acid that codes for at least one of: a therapeutic protein or a diagnostic protein. In some embodiments, the exogenous nucleic acid can code for at least one of: a chemokine receptor, a membrane associated protein, a microbial protein that is capable of degrading hyaluronan, a microbial protein, SOCS3, PH-20, HMGB1, PIAS3, IL15, IL15-Rα, LIGHT, ITAC, fractalkine, NIL, an immune checkpoint modulator, a metabolic modulating protein, or any combinations thereof, such as a fusion protein comprising any combination of the above (such as a metabolic modulating protein and a cytokine), In some embodiments, the exogenous nucleic acid that codes for a chemokine receptor, wherein the chemokine receptor comprises at least one of CXCR4 and CCR2. In some embodiments, the exogenous nucleic acid that codes for the membrane associated protein. In some embodiments, the membrane associated protein comprises a membraned associated hyaluronidase. In some embodiments, the membrane associated hyaluronidase comprises PH-20. In some embodiments, the PH-20 is GP1-anchored. In some embodiments, the exogenous nucleic acid that codes for the microbial protein that is capable of degrading hyaluronan, wherein the microbial protein comprises a secreted hyaluronidase. In some embodiments, the secreted hyaluronidase comprises at least one of HysA, lin, sko, and rv, or any combinations thereof. In some embodiments, the exogenous nucleic acid that codes for the microbial protein. In some embodiments, the microbial protein comprises HysA. In some embodiments, the modified oncolytic poxvirus further comprises a modification in the genome of the virus, wherein the modification comprises a mutation or a deletion of the B5R gene. In some embodiments, the modification in the genome of the virus, wherein the modification comprises a mutation or a deletion in a SCR region of the B5R gene, wherein said SCR region comprises SCR1, SCR3, SCR4, or any combinations thereof, and wherein the SCR region does not comprise SCR2. In some embodiments, the modified oncolytic poxvirus further comprises a mutation or a deletion of a viral gene selected from a group consisting of: Thymidine kinase (TK), B8R, B18R, B15R, K7R, C6L, K4L, F8L, F9L, F10L, F17R, E1L, E4L, E6R, E8R, E10R, E11L, O2L, I1L, I2L, I3L, I5L, I7L, I8R, G1L, G3L, G4L, G5.5R, C7L, G9R, L1R, L3L, L4R, L5R, J1R, J4R, J6R, H1L, H2R, H3L, H4L, H6R, D1R, D2L, D3R, D6R, D7R, D8L, D11L, D12L, D13L, A2.5L; A3L, A4L, A5R, A6L, A7L, A9L, A10L, A13L, A14L, A15L, A17L, A18R, A21L, A24R, A25L, A26L, A27L, A28L, A29L, A30L, A31R, A34R, A42R, A45R, A46R, A52R, and any combinations thereof. In some embodiments, the modified oncolytic poxvirus comprises the mutation or deletion of viral gene A52R. In some embodiments, the modified oncolytic poxvirus comprises (i) the exogenous nucleic acid that codes for a chemokine receptor, wherein the chemokine receptor comprises at (ii) the exogenous nucleic acid that codes for PIAS3; (iii) a mutation or deletion of the thymidine kinase gene; (iv) the mutation or deletion of the A52R gene. In some embodiments, the virus is suitable for systemic delivery. In some embodiments, the virus is capable of immune evasion. In some embodiments, the systemic delivery comprises oral administration; parenteral administration, intranasal administration, sublingual administration, rectal administration, transdermal administration, or any combinations thereof. In some embodiments, the parenteral administration comprises an intravenous injection. In some embodiments, the virus is suitable for intratumoral delivery. In some embodiments, the poxvirus is a vaccinia virus.

One embodiment provides a process for engineering an oncolytic poxvirus comprising: (i) obtaining an oncolytic poxvirus DNA backbone vector, the oncolytic poxvirus DNA backbone vector comprising one or more modifications according to any one of the preceding claims; (ii) further modifying the oncolytic virus DNA vector to produce an engineered DNA vector; (iii) transfecting mammalian cells with the engineered. DNA vector; (iv) culturing the mammalian cells under conditions suitable for viral replication; and (v) harvesting the viral particles.

In some embodiments, the mammalian cells comprise HeLa cells, 293 cells, A549 cells, or Vero cells.

One embodiment provides a kit, comprising: the oncolytic poxvirus, a container; and instructions for administering said oncolytic virus to a subject to treat a disorder associated with pathological angiogenesis.

One embodiments provides a method of treating a tumor, the method comprising administering to a subject a therapeutically effective amount of the oncolytic poxvirus according.

One embodiments provides a method of treating a tumor, the method comprising administering to a subject a composition comprising patient-derived leukocyte cells infected with a modified oncolytic poxvirus that expresses an A34R protein comprising mutations at positions 119 and 151 of the wild-type A34 protein (SEQ ID No. 4), wherein the modified oncolytic poxvirus produces a population of viral particles in a tumor microenvironment. In some embodiments, the patient-derived leukocyte cells comprise macrophages. In some embodiments, the patient-derived leukocyte cells comprise tumor-targeted T cells. In some embodiments, at least about 10% to at least about 90% of the population of viral particles are EEV particles, as measured in a viral plaque assay. In some embodiments, the method further comprises harvesting the EEV particles from the tumor microenvironment and intravenously administering the EEV particles to the subject. In some embodiments, the modified oncolytic poxvirus is a modified oncolytic vaccinia virus.

One embodiment provides a process comprising infecting a culture of host cells with a population of modified oncolytic poxvirus that comprises at least about 10% to at least about 90% EEV particles, wherein the modified oncolytic poxvirus expresses an A34R protein comprising mutations at positions 119 and 151 of the wild-type A34 protein (SEQ ID No. 4). In some embodiments, the modified oncolytic poxvirus is a modified oncolytic vaccinia virus.

One embodiment provides a method of treating a cancer, the method comprising administering to a patient a modified oncolytic virus comprising a nucleic acid that codes for an A34R protein or a fragment thereof comprising at least two mutations, wherein the at least two mutations are in positions corresponding to positions Lys119 and Lys151 of a wild-type vaccinia virus A43R protein (SEQ ID NO. 4). In some embodiments, the at least two mutations n positions corresponding to positions Lys119 and Lys151 of a wild-type vaccinia virus A43R protein (SEQ ID NO. 4) are Lys119Glu and Lys151Glu, respectively-.

One embodiment provides a method of treating a tumor, the method comprising administering to a patient a modified oncolytic virus comprising a nucleic acid that codes for an A34R protein or a fragment thereof comprising at least two mutations, wherein the at least two mutations are in positions corresponding to positions Lys119 and Lys151 of a wild-type vaccinia virus A43R protein (SEQ ID NO. 4). In some embodiments, the at least two mutations n positions corresponding to positions Lys119 and Lys151 of a wild-type vaccinia virus A43R protein (SEQ ID NO. 4) are Lys119Glu and Lys151Glu, respectively.

In some embodiments, the administering is via an intratumoral injection, an intravenous injection, or a combination thereof. In some embodiments, the administering is via an intratumoral injection, an intravenous injection, or a combination thereof.

In some embodiments, the method further comprises administering a further therapy, in combination with the oncolytic poxvirus, wherein the further therapy comprises at least one of: a chemotherapy, a radiation therapy, an oncolytic viral therapy with an additional virus, treatment with an immunomodulatory protein, a CAR T cellular therapy, an anti-cancer agent, an immunomodulatory agent, or any combinations thereof.

In some embodiments, the further therapy comprises the immunomodulatory agent selected from the group consisting of: an anti-CD33 antibody or an antigen binding fragment thereof, an anti-CD11b antibody or an antigen binding fragment thereof, a COX2 inhibitor, a cytokine, a chemokine, an anti-CTLA4 antibody or an antigen binding fragment thereof, an anti-PD-1 antibody or an antigen binding fragment thereof, an anti-PD-L1 antibody or an antigen binding fragment thereof, and a TLR agonist.

### BRIEF DESCRIPTION OF THE DRAWINGS

The novel features of the disclosure are set forth with particularity in the appended claims. A better understanding of the features and advantages of this disclosure will be obtained by reference to the following detailed description that sets forth illustrative embodiments, in which the principles of this disclosure are utilized, and the accompanying drawings of which.

FIG. 1 shows an exemplary assembly scheme to generate DNA for making a recombinant virus library.

FIG. 2 shows a comparison of viral plaque comet tails formed by different vaccinia virus strains.

FIGS. 3A-3B show results of neutralization assay carried out using different vaccinia virus strains (FIG. 3A shows results following treatment with NR-45114 antibody and FIG. 3B shows results following treatment with anti-L1R and VIG antibodies).

FIG. 4 shows results cell viability (upper panel: MC38 cells; lower panel: HCT116 cells) following infection with different vaccinia virus strains.

FIG. 5 shows results of a viral replication assay in cancer cells (upper panel: HCT116 cells; lower panel: MC38 cells), for different vaccinia virus strains.

### DETAILED DESCRIPTION

While preferred embodiments of this disclosure have been shown and described herein, it will be obvious to those skilled in the art that such embodiments are provided by way of example only. Numerous variations, changes, and substitutions will now occur to those skilled in the art without departing from this disclosure. It should be understood that various alternatives to the embodiments of this disclosure described herein may be employed in practicing the disclosure. It is intended that the following claims define the scope of the disclosure and that methods and structures within the scope of these claims and their equivalents be covered thereby.

### Certain Definitions

The terminology used herein is for the purpose of describing particular cases only and is not intended to be limiting. As used herein, the singular forms “a”, “an” and “the” can include the plural forms as well, unless the context clearly indicates otherwise. Furthermore, to the extent that the terms “contains,” “containing,” “including”, “includes,” “having,” “has”, “with”, or variants thereof are used in either the detailed description and/or the claims, such terms are intended to be inclusive in a manner similar to the term “comprising.”

The term “about” or “approximately” can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value, such as ±10% of the value modified by the term “about”.

The terms “individual,” “patient,” or “subject” can be used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker). In some embodiments, patients, subjects, or individuals can be under the supervision of a health care worker.

The terms “heterologous nucleic acid sequence,” or “exogenous nucleic acid sequence,” or “transgenes,” as used herein, in relation to a specific virus can refer to a nucleic acid sequence that originates from a source other than the specified virus.

The term “mutation,” as used herein, can refer to a deletion, an insertion of a heterologous nucleic acid, an inversion or a substitution, including an open reading frame ablating mutations as commonly understood in the art.

The term “gene,” as used herein, can refer to a segment of nucleic acid that encodes an individual protein or RNA (also referred to as a “coding sequence” or “coding region”), optionally together with associated regulatory regions such as promoters, operators, terminators and the like, which may be located upstream or downstream of the coding sequence.

The terms “mutant virus” and “modified virus,” as used interchangeably herein, can refer to a virus comprising one or more mutations in its genome, including but not limited to deletions, insertions of heterologous nucleic acids, inversions, substitutions or combinations thereof.

The term. “naturally-occurring,” as used herein with reference to a virus, can indicate that the virus can be found in nature, i.e., it can be isolated from a source in nature and has not been intentionally modified, e.g., a wild-type virus.

The term “non-naturally-occurring,” as used herein with reference to one or more mutations in viral nucleic acid sequences or in the amino acid sequence of viral proteins, can indicate that a viral strain comprising the one or more mutations cannot be found in nature, i.e., it cannot be isolated from a source in nature and has instead been intentionally modified.

The terms “inhibiting,” “reducing” or “prevention,” or any variation of these terms, referred to herein, can include any measurable decrease or complete inhibition to achieve a desired result.

A “promoter,” as used herein, can be a control sequence that is a region of a nucleic acid sequence at which initiation and rate of transcription are controlled. In certain embodiments, a promoter may contain genetic elements at which regulatory proteins and molecules may bind such as RNA polymerase and other transcription factors. The terms “operatively positioned.” “operatively linked,” “under control” and “under transcriptional control” can mean that a promoter is in a correct functional location and/or orientation in relation to a nucleic acid sequence to control transcriptional initiation and/or expression of that sequence. In certain embodiments, a promoter may or may not be used in conjunction with an “enhancer,” which refers to a cis-acting regulatory sequence involved in the transcriptional activation of a nucleic acid sequence.

The term “homology,” as used herein, may be to calculations of “homology” or “percent homology” between two or more nucleotide or amino acid sequences that can be determined by aligning the sequences for optimal comparison purposes (e.g., gaps can be introduced in the sequence of a first sequence). The nucleotides at corresponding positions may then be compared, and the percent identity between the two sequences may be a function of the number of identical positions shared by the sequences (i.e., % homology=# of identical positions/total # of positions×100). For example, a position in the first sequence may be occupied by the same nucleotide as the corresponding position in the second sequence, then the molecules are identical at that position. The percent homology between the two sequences may be a function of the number of identical positions shared by the sequences, taking into account the number of gaps, and the length of each gap, which need to be introduced for optimal alignment of the two sequences. In some embodiments, the length of a sequence aligned for comparison purposes may be at least about: 30%, 40%, 50%, 60%, 65%, 70?, 75%, 80%, 85?, 90%, 91%, 92%, 93%, 94%, 95%, 96%, 97%, 98%, or 95%, of the length of the reference sequence. A BLAST® search may determine homology between two sequences. The homology can be between the entire lengths of two sequences or between fractions of the entire lengths of two sequences. The two sequences can be genes, nucleotides sequences, protein sequences, peptide sequences, amino acid sequences, or fragments thereof. The actual comparison of the two sequences can be accomplished by well-known methods, for example, using a mathematical algorithm. A non-limiting example of such a mathematical algorithm may be described in Karlin, S. and Altschul, S., Proc. Natl. Acad. Sci. USA, 90-5873-5877 (1993), Such an algorithm may be incorporated into the NBLAST and XBLAST programs (version 2.0), as described in Altschul, S. et al., Nucleic Acids Res., 25:3389-3402 (1997). When utilizing BLAST and Gapped BLAST programs, any relevant parameters of the respective programs (e.g., NBLAST) can be used. For example, parameters for sequence comparison can be set at score=100, word length=12, or can be varied (e.g., W=5 or W=20). Other examples include the algorithm of Myers and Miller, CABIOS (1989), ADVANCE, ADAM, BLAT, and FASTA. In another embodiment, the percent identity between two amino acid sequences can be accomplished using, for example, the GAP program in the GCG software package (Accelrys, Cambridge, UK).

The term “subject” can refer to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

The terms “treat,” “treating,” and “treatment” can be meant to include alleviating or abrogating a disorder, disease, or condition; or one or more of the symptoms associated with the disorder, disease, or condition; or alleviating or eradicating the cause(s) of the disorder, disease, or condition itself. Desirable effects of treatment can include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

The term “therapeutically effective amount” can refer to the amount of a compound that, when administered, can be sufficient to prevent development of, or alleviate to some extent, one or more of the symptoms of the disorder, disease, or condition being treated. The term “therapeutically effective amount” can also refer to the amount of a compound that is sufficient to elicit the biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” can refer to a pharmaceutically-acceptable material, composition, or vehicle, such as a liquid or solid filler, diluent, excipient, solvent, or encapsulating material. A component can be “pharmaceutically acceptable” in the sense of being compatible with the other ingredients of a pharmaceutical formulation. It can also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed.; CRC Press LLC: Boca Raton, Fla., 2004).

The term “pharmaceutical composition” can refer to a mixture of a compound disclosed herein with other chemical components, such as diluents or carriers. The pharmaceutical composition can facilitate administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid; ethanesulfonic acid, p-toluenesulfonic acid, salicylic acid and the like.

An “anti-cancer agent,” as used herein, can refer to an agent or therapy that is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. Non-limiting examples of anti-cancer agents can include biological agents (biotherapy), chemotherapy agents, and radiotherapy agents.

The term “oncolytic,” as used herein, can refer to killing of cancer or tumor cells by an agent, such as an oncolytic virus, such as an oncolytic poxvirus, such as an oncolytic vaccinia virus, e.g., through the direct lysis of said cells, by stimulating immune response towards said cells, apoptosis, expression of toxic proteins, autophagy and shut-down of protein synthesis, induction of anti-tumoral immunity, or any combinations thereof. The direct lysis of the cancer or tumor cells infected by the agent, such as an oncolytic vaccinia virus, can be a result of replication of the virus within said cells. In certain examples, the term “oncolytic,” can refer to killing of cancer or tumor cells without lysis of said cells.

The term “oncolytic virus” as used herein can refer to a virus that preferentially infects and kills tumor cells. Linder certain non-limiting circumstances, it is understood that oncolytic viruses can promote anti-tumor responses through dual mechanisms dependent on not only the selective killing of tumor cells, but also the stimulation of host anti-tumor immune responses.

In some embodiments, the oncolytic viruses can include, but are not limited to, (i) viruses that naturally replicate preferentially in cancer cells and are non-pathogenic in humans often due to elevated sensitivity to innate antiviral signaling or dependence on oncogenic signaling pathways; and (ii) viruses that are genetically-manipulated for use. In some embodiments, an oncolytic virus can comprise a herpes simplex virus (HSV). In some embodiments, an oncolytic virus can comprise a poxvirus. In some embodiments a poxvirus can comprise a leporipoxvirus or a vaccinia virus. In some embodiments, a vaccinia virus can comprise a vaccinia virus of the, Ankara strain, Western Reserve strain (WR), or the Copenhagen strain. In some embodiments a vaccinia virus can comprise a Lister, Wyeth, New York City Board of Health, Tian Tan, Tash Kent, or USSR strain. In some embodiments a leporipoxvirus can comprise a myxoma virus. In some embodiments an oncolytic virus can be modified.

The term “modified oncolytic virus” as used herein can refer to an oncolytic virus that comprises a modification to its constituent, such as, but not limited to, a modification in the native genome (“backbone”) of the virus like a mutation or a deletion of a viral gene, introduction of an exogenous nucleic acid, a chemical modification of a viral nucleic acid or a viral protein, and introduction of a exogenous protein or modified viral protein to the viral capsid. In general, oncolytic viruses may be modified (also known as “engineered”) in order to gain improved therapeutic effects against tumor cells. In certain embodiments, the modified oncolytic virus can be a modified pox virus. In certain embodiments, the modified oncolytic virus can be a modified pox virus.

The terms “systemic delivery,” and “systemic administration,” used interchangeably herein, in some cases can refer to a route of administration of medication, oncolytic virus or other substances into the circulatory system. The systemic administration may comprise oral administration, parenteral administration, intranasal administration, sublingual administration, rectal administration, transdermal administration, or any combinations thereof.

### Oncolytic Vaccinia Viruses in Extracellular Enveloped Virus Form

Poxviruses (such as vaccinia viruses) can exist in several forms, including the IMV (Intracellular Mature Virus; which is highly antigenic but stable, and can be important for host to host spread of poxviruses), and the EEV (Extracellular Enveloped Virus; which can be unstable outside a host, but may be capable of enhanced spread within the host due to a host cell derived outer envelope that conceals the virus; as such, the EEV form can be helpful for systemic spread of the vaccinia virus, e.g., the oncolytic vaccinia virus within a host).

Different strains of Vaccinia are known to produce different ratios of IMV and EEV particles subsequent to infection of a susceptible cell, with Western Reserve (WR) strain being a low EEV producing strain, and International Health Department (IHD)-J strain of vaccinia (IHD-J) being a high EEV producing strain. One example of a point mutation in a vaccinia gene that is present in IHD-J, but not in WR, is in the A34R protein (K151E), which is encoded by the vaccinia virus gene VACWR157. A strain derived from WR, containing this mutation (WI strain, which is a WR virus with the A34R gene from IHD-J recombined into the WR A34R gene locus) was shown to increase EEV production (See Blasco, R., et al 1993 J. Virol. June; 67(6):3319-25).

In some embodiments of this disclosure, provided is a modified oncolytic poxvirus (e.g., a modified oncolytic vaccinia virus strain) that can comprise a modification, such as a non-naturally occurring mutation in a viral glycoprotein (e.g., A34R protein; wild type sequence provided in UniProt Accession No. P24761; SEQ ID No. 4), that enhances the ratio of extracellular enveloped virus (EEV) to intracellular mature virus (IMV) form of the virus. For instance, the modified oncolytic vaccinia virus strain comprising the non-naturally occurring mutation in the viral glycoprotein (e.g., A34R protein) can release a higher amount of EEV particles, compared to IMV particles.

In some embodiments are provided a modified oncolytic vaccinia virus strain which may comprise two or more non-naturally occurring mutations in a viral glycoprotein, such as A34R. Exemplary amino acid sequence for the mutated A34R protein (also referred to herein as “WO34”) is provided in SEQ ID No. 5.

In some embodiments, the two or more non-naturally occurring mutations can be at positively charged amino acid residues (e.g., lysine) within the wild-type A34R protein (SEQ ID No. 4), or a fragment thereof, wherein the positive charged amino acid residues are positively charged at pH 5. In some embodiments, at least one of the two or more non-naturally occurring mutations can be at position 110 within the wild-type A34R protein (SEQ ID No. 4), or a fragment thereof. In some examples, if at least one of the two or more mutations is at position 110 of the wild-type A34R protein (SEQ ID No. 4), then the mutated amino acid at that position is not asparagine.

In some embodiments, the two or more non-naturally occurring mutations are not in aspartic acid residues within the wild-type A34R protein (SEQ ID No. 4), or a fragment thereof. In some embodiments, the two or more non-naturally occurring mutations can independently be in alanine, arginine, asparagine, cysteine, glutamine, glutamic acid, glycine, histidine, isoleucine, leucine, lysine, methionine, phenylalanine, proline, serine, threonine, tryptophan, tyrosine, or valine residues within the wild-type A34R protein (SEQ ID No. 4), or a fragment thereof. In some embodiments, the A34R protein or a fragment thereof, expressed by the modified oncolytic pox virus can comprise a non-naturally occurring mutation in a lysine residue, wherein the lysine residue is at a position other than position Lys151 of the wild-type A34R protein (SEQ ID No. 4) or a fragment thereof. In some embodiments, the A34R protein or a fragment thereof expressed by the modified oncolytic poxvirus can comprise a non-naturally occurring mutation at position Lys11.9.

In some embodiments the two or more non-naturally occurring mutations may be at residues 119 and 151 of the wild-type A34R protein (SEQ ID No. 4), or a fragment thereof. In some embodiments, the mutation at position 119 can be a Lys119Glu (K119E). In some embodiments, the mutation at position 151 can be Lys151Glu (K151E).

In some embodiments are provided a modified oncolytic poxvirus strain which may comprise mutations in viral proteins such as haemaglutinin, neuraminidase, Spike(S) glycoprotein, E1, E2, gp120, gp160, gp41, gp1, gp2, E (dimer). E1, or E2.

In some cases, the two or more non-naturally occurring mutations in the modified oncolytic poxvirus A34R protein may result in an increase in the ratio of extracellular enveloped virus (EEV) form to intracellular mature virus (IMV) form of the virus.

In some embodiments the two or more non-naturally occurring mutations in the modified oncolytic poxvirus A34R protein may result in an increase in a ratio of EEV form to IMV form of the virus in comparison to a poxvirus strain that does not comprise the two or more non-naturally occurring mutations in the A34R protein, but is otherwise identical.

In some embodiments, a modified poxvirus strain provided herein, comprising at least two non-naturally occurring mutations in the A34 protein, releases high levels of EEV particles, as measured by increased comet formation in tissue culture, compared to large round plaques in tissue culture formed by poxvirus strains that release lower levels of EEV particles in tissue culture (e.g., a vaccinia virus strain that does not comprise at least two non-naturally occurring imitations in the A34R protein).

The EEV particles released by the modified poxvirus of this disclosure, comprising at least two non-naturally occurring mutations in the A34R protein are, in some embodiments, resistant to neutralization by antibodies (neutralizing antibodies), and complement toxicity, while the IMV particles are not. As such, the FEY particles may mediate long range dissemination in vitro and in vivo.

The EEV particles can also have a higher specific infectivity in comparison to EEV particles (as determined by a lower particle/pfu ratio). Accordingly, the modified poxvirus strain releasing higher levels of FEY particles can be an improved virus for therapeutic use.

In some embodiments, certain host-cell derived proteins can co-localize with EEV preparations, but not with IMV, and the amount of cell-derived proteins can be dependent on the host cell line and the virus strain. For instance, a study has shown that the WR EEV contains more cell-derived proteins in comparison to VV IHD-J strain (See van Eijl H, Hollinshead M, Smith G L. The vaccinia virus A36R protein is a type 1b membrane protein present on intracellular but not extracellular enveloped virus particles, Virology 2000; 271: 26-36). In some cases, host cell derived proteins can modify biological effects of EEV particles. As an example, incorporation of the host membrane protein CD55 in the surface of the EEV particles released by the WR vaccinia virus strain comprising at least two non-naturally occurring mutations in the A34R protein, can make them resistant to complement toxicity.

For Western Reserve (WR) strain of vaccinia virus, about 1% of virus particles are normally FEY and are released into the culture supernatant before oncolysis of the producer cell. Some studies have shown that 50-fold more EEV particles may be released from an strain of vaccinia (See Blasco R, Sisler J R, Moss B. Dissociation of progeny vaccinia virus from the cell membrane is regulated by a viral envelope glycoprotein: effect of a point mutation in the lectin homology domain of the A34R gene, J Virol 1993; 67:33_19-25 see also Mcintosh A A, Smith G L. Vaccinia virus glycoprotein A34R is required for infectivity of extracellular enveloped virus. J Virol 1996; 70: 272-81).

A modified pox virus (e.g., vaccinia virus) strain of this disclosure, can, in some examples, release about 10-fold to about 200-fold higher levels of EEV particles, compared to a poxvirus strain which is otherwise identical but does not comprise an A34R protein comprising at least two non-naturally occurring mutations (e.g., K151E and K119E).

In some embodiments, a modified poxvirus strain of this disclosure can release about 10-fold to about 15-fold, about 15-fold to about 20-fold, about 20-fold to about 25-fold, about 25-fold to about 30-fold, about 30-fold to about 35-fold, about 35-fold to about 40-fold, about 40-fold to about 45-fold, about 45-fold to about 50-fold, about 50-fold to about 55-fold, about 55-fold to about 60-fold, about 60-fold to about 65-fold, about 65-fold to about 70-fold, about 75-fold to about 80-fold, about 85-fold to about 90-fold, about 95-fold to about 100-fold, about 100-fold to about 120-fold, about 120-fold to about 140-fold, about 140-fold to about 160-fold, about 160-fold to about 180-fold, about 180-fold to about 200-fold more EEV particles compared to a poxvirus strain which is otherwise identical but does not comprise an A34R protein comprising at least two non-naturally occurring mutations (e.g., K151E and K119E).

In some embodiments, a modified vaccinia virus strain of this disclosure can be a WR strain wherein the A34R protein comprises the mutations K119E and K151E, and can release about 10-fold to about 15-fold, about 15-fold to about 20-fold, about 20-fold to about 25-fold, about 25-fold to about 30-fold, about 30-fold to about 35-fold, about 35-fold to about 40-fold, about 40-fold to about 45-fold, about 45-fold to about 50-fold, about 50-fold to about 55-fold, about 55-fold to about 60-fold, about 60-fold to about 65-fold, about 65-fold to about 70-fold, about 75-fold to about 80-fold, about 85-fold to about 90-fold, about 95-fold to about 100-fold, about 100-fold to about 120-fold, about 120-fold to about 140-fold, about 140-fold to about 160-fold, about 160-fold to about 180-fold, about 180-fold to about 200-fold more EEV particles compared to a WR vaccinia virus strain which is otherwise identical but does not comprise an A34R protein comprising the mutations K119E and K151E.

In some embodiments, a modified vaccinia virus strain of this disclosure can be a WR strain wherein the A34R protein comprises the mutations K119E and K151E (WO34), and can release about 10-fold to about 15-fold, about 15-fold to about 20-fold, about 20-fold to about 25-fold, about 25-fold to about 30-fold, about 30-fold to about 35-fold, about 35-fold to about 40-fold, about 40-fold to about 45-fold, about 45-fold to about 50-fold, about 50-fold to about 55-fold, about 55-fold to about 60-fold, about 60-fold to about 65-fold, about 65-fold to about 70-fold, about 75-fold to about 80-fold, about 85-fold to about 90-fold, about 95-fold to about 100-fold, about 100-fold to about 120-fold, about 120-fold to about 140-fold, about 140-fold to about 160-fold, about 160-fold to about 180-fold, about 180-fold to about 200-fold more EEV particles compared to a WI vaccinia virus strain.

In some cases an increase in release of EEV particle, compared to IMV particle, by a modified poxvirus of this disclosure may be determined by performing a viral plaque assay, wherein a greater number of comet-tail formations may be observed in a poxvirus comprising the two or more non-naturally occurring mutations in the A34R protein, in comparison to a poxvirus that does not comprise the two or more mutations in the A34R protein, but is otherwise identical.

In some cases an increase in release of EEV particle, compared to IMV particle, may be determined by performing a neutralization assay, wherein cells infected with a modified poxvirus (e.g., a vaccinia virus strain) of this disclosure and exposed to neutralizing antibodies (such as anti-L1 NR-45114 antibody or VIG antibody), can be tested in a viral plaque assay, and viral plaque formation (e.g., in PFU/mL) can be compared with appropriate control viruses (e.g., a vaccinia virus strain that does not comprise at least two non-naturally occurring mutations in the A34R protein), In some cases anti-Lt can neutralize and block IMV infection. In some cases VIG antibody can block EEV infection.

In some cases an increase in release of EEV particles compared to IMV particles may be determined by observing an increase in comet tail formation. In some cases observing an increase in comet tail formation can comprise counting the number of colonies on a plate with a comet tail appearance generated from a known amount of virus that has been plated and comparing with the number on a plate with another equivalent amount of virus plated. In some cases an increase in the formation of comets can indicate an increase in the amount of EEV relative to IMV forms of a viral strain.

In some cases a modified poxvirus strain of this disclosure can comprise one or more additional mutations in a region of the viral genome that codes for a phospholipase, a kinase, a phosphoprotein, a polymerase, a membrane protein, a virion core protein, a glutaredoxin, a DNA binding protein, an RNA binding protein, an IMV protein, a proteinase, a helicase, a metalloproteinase, a virion structural protein, a myristyl protein, a phosphatase, a heparin binding protein, a glycoprotein, an ATPase, a capping enzyme, a transcription factor, a precursor protein, a subunit protein, a DNA helicase, a palmityl protein, or a receptor.

In some cases one or more additional mutations may be in a poxviral gene, such as TK (thymidine kinase), B8R, B18R, B15R, K7R, C6L, K4L, F8L, F9L, F10L, F17R, E1L, E4L, E6R, E8R, E10R, E11L, O2L, I1L, I2L, I3L, I5L, I7L, I8R, G1L, G3L, G4L, G5.5R, G7L, G9R, L3L, L4R, L5R, J1R, J4R, J6R, H1L, H2R, H3L, H4L, H5R, H6R, D1R, D2L, D3R, D6R, D7R, D8L, D11L, D12L, D13L, A2.5L, A31L, A4L, A5R, A6L, A7L, A9L, A10L, A13L, A14L, A15L, A16L, A17L, A18R, A21R, A24R, A25L, A26L, A27L, A28L, A29L, A30L, A31R, A34R, A42R, A45R, A46R, A52R gene of a poxvirus.

Also provided herein, in some embodiments, is a modified oncolytic poxvirus (e.g., a vaccinia virus strain that can comprise non-naturally occurring mutations that increase the EEV form of the virus, and further, an exogenous nucleic acid that can code for a non-viral protein, such as a therapeutic protein or a diagnostic protein. Non-limiting examples of a protein encoded by the exogenous nucleic acid can include SOC3, PH-20, HMGB1, PIAS3, IL15, IL15-Rα, LIGHT, ITAC, fractalkine, CXCR4, CCR2, CCL5, N1L, an immune checkpoint modulator (e.g., an anti-PD-1 antibody, an anti-PD-L1 antibody, an anti-CTLA4 antibody), GM-CSF, IL-12, IL-2, INS, EPO, env, F8, GCG, IFNG, IGHG1, F9, GH1, IL-2, CSF2, TNFRSF1B, ALB, PLAU, IFNB1, CSF3, IFNA2, FSHB, Botulinum Toxin Type A, Alefacept, Pancrelipase, Antithrombin Alfa, Arcitumornab, Anti-rhesus (rh) immunoglobulin G, anti-thymocyte globulin, alemtuzurnab, abciximab, alglucosidase alfa, abatacept, pegademase, apcitide, human serum albumin, rasburicase, bevacizumab, botulinum toxin type B, bivalirudin choriogonadotrophin alfa, pegfilgrastim, collagenase clostridium histiolyticum, filgrastim, crotalidae polyvalent immune Fab, Sargramostim, dornase alfa, deniletikin diftitox, digoxin immune fab, epoetin alfa, darbepoetin alfa, eculizumab, enfuvirtide, exenatide, efalizumab, palifermin, coagulation factor IX, antihemophilic factor, thyrotropin alfa, follitropin beta, coagulation factor Vila recombinant human, gemtuzumab ozogamicin, galsulfase, pegvisomant, imiglucerase, somatropin, glucagon, agalsidase beta, alglucerase, hyaluronidase, histrehn, hepatitis C antigens, HIV antigens, hepatitis B surface antigen, HPV vaccine, hyaluronidase, interferon alfa-2b, interferon gamma-1b, insulin, laronidase, ibritumomab tiuxetan, insulin, infliximab, interferon beta-1b, oprelvekin, idursulfase, panitumumah, immune globulin human, cetuximab, adalimumab, pegaspargase, daclizumah, asparaginase, interferon alfacon-1, interferon alfa-n3, lutropin alfa, lepirudin, lactase, muromonab, mecasermin, natalizumab, nofetumomah, nesiritide, octreotide, ospA lipoprotein, tenecteplase, pramlintide, papain, urokinase, anistreplase, drotrecogin reteplase, becaplermin, palivizumab, alteplase, ranibizumab, recombinant human bone morphogenic preotein 7 (rhBMP7), recombinant purified protein derivative (DPPD), streptokinase, calcitonin, sennorelin, secretin, alpha-1-proteinase inhibitor, satumomab pendetide, technetium fanolesomab, teriparatide, trypsin, etanercept, and functional domains or fragments or variants thereof, or any combinations thereof.

Hyaluronan (HA) is an important structural element of ECM and a high molecular weight linear glycosaminoglycan consisting of repeating disaccharide units. It can be distributed widely throughout connective, epithelial, and neural tissues, and its expression level can be significantly elevated in many types of tumors. Hyaluronidases are a family of enzymes that catalyze the degradation of HA. There are at least five functional hyaluronidases identified so far in human: HYAL1. HYAL2, HYAL3, HYAL4 and HYAL5 (also known as PH-20 or SPAM1), among which PH-20 is the only one known so far to be functional at relatively neutral pH. In some embodiments of the present disclosure, combining hyaluronidase with other tumor-targeting therapeutic agents (such as transgenes, also referred to herein as exogenous nucleic acid) can promote the therapeutic effect of the modified oncolytic virus at least by diminishing the ECM and enhancing the transportation of the therapeutic agent inside and between the tumors.

Some embodiments herein disclose a modified oncolytic virus that can comprise an exogenous nucleic acid coding for a membrane associated protein that is capable of degrading hyaluronan, such as a hyaluronidase. It should be noted that the term “hyaluronidase” as used herein can refer to any enzyme or a fragment thereof that catalyzes the degradation of HA in a tumor, including, but not limited to, PH-20 and its homologs from other species, as well as other engineered/design proteins with similar enzymatic function. As used herein, hyaluronidase can refer to a class of hyaluronan degrading enzymes.

In some embodiments, the modified oncolytic virus comprises an exogenous nucleic acid that can code for a chemokine receptor that is a chimeric protein. At least part of its extracellular domain can be from a chemokine receptor that promotes the tumor-targeted delivery of the virus, and at least part of its intracellular domain can be from a chemokine receptor that promotes the tumor-specific replication, inhibits immunosuppressive activity, or conveys some other beneficial effects, or vice versa. For instance, the modified oncolytic virus can comprise a nucleic acid that codes for a protein having an intracellular GTPase domain of CCR5, and an extracellular chemokine-binding domain of CXCR4 or CCR2. In some case, by combining domains with different functionalities one may achieve further improvement in therapeutic performance of the modified oncolytic virus. It is one embodiment of this disclosure that the modified oncolytic virus can comprise exogenous nucleic acids that can code for at least one chemokine receptor. In some cases, the modified oncolytic virus can comprise exogenous nucleic acids that can code for two or more different chemokine receptors, which may be expressed simultaneously by the virus. Exemplary chemokine receptors that can be expressed simultaneously from the modified oncolytic viruses described herein can include CXCR4 and CCR2. In modified oncolytic viruses expressing more than one chemokine receptors, a combinatorial or synergistic effect against tumor cells may be achieved as to the therapeutic application of the oncolytic virus.

In certain embodiments, the modified oncolytic virus comprises an exogenous CXCR4-expressing nucleic acid. In certain embodiments, the modified oncolytic virus comprises an exogenous CCR2-expressing nucleic acid. Certain embodiments disclose a modified oncolytic virus comprising an exogenous nucleic acid that codes for both CXCR4 and CCR2, and both chemokines are expressed form the same virus. Under certain circumstances, CXCL12 and/or CC12 typically expressed in the tumor microenvironment may attract the CXCR4 and/or CCR2-expressing lymphocytes or other migrating cells that are infected by the modified oncolytic virus, thereby enhancing the tumor-targeted delivery of the modified oncolytic virus.

In certain embodiments, modified viruses described herein can comprise one or more exogenous nucleic acid sequences, alternatively referred to as transgenes, which can generate mRNAs coding for an agent that can modulate the activity of STAT3 and as a result can also modulate the activation of genes regulated by STAT3. Thus, certain examples provided herein provide oncolytic vaccinia viruses containing exogenous nucleic acid sequences that can encode an agent that can modulate STAT-3 mediated gene-activation. The phrase “modulates STAT 3-mediated gene activation,” as used herein, can refer to a process wherein STAT3 activity is modulated and as a consequence the activation of one or more genes that are regulated by STAT3 is also modulated.

In certain embodiments, the agent that can modulate STAT3-mediated gene activation can be a protein or a fragment thereof. In certain embodiments, the protein or the fragment thereof can inhibit, reduce, or minimize STAT3 activity and STAT3-mediated gene activation. A protein or a fragment thereof that inhibits, reduces and/or minimizes STAT3 activity and STAT3-mediated gene activation can, for example, block the binding of STAT3 to a DNA binding sequence in the promoter regions of STAT3 responsive genes. In additional examples, the protein or a fragment thereof that inhibits, reduces, or minimizes STAT3 activity and STAT3-mediated gene activation can directly bind the STAT3 protein, for example, at the SW domain. In certain embodiments, a protein that inhibits, reduces and/or minimizes STAT3 activity blocks, prevents, reduces and/or minimizes the phosphorylation of STAT3 and/or dephosphorylates STAT3. In certain non-limiting embodiments, the proteins that modulate STAT3 activity can include phosphotyrosine phosphatases (PTPs), protein inhibitor of activated STAT (PIAS, e.g., PIAS3) and suppressor of cytokine signaling (SOLS) proteins (e.g., SOC3).

### Cancer Targets

In an embodiment of this disclosure, a method of treatment for a hyperproliferative disease, such as a cancer or a tumor, by the delivery of a modifiesd oncolytic poxvirus as described herein, is provided. Cancers that can be treated by a modified oncolytic poxvirus, as described herein, can include, but are not limited to, melanoma, hepatocellular carcinoma, breast cancer, lung cancer, peritoneal cancer, prostate cancer, bladder cancer, ovarian cancer, leukemia, lymphoma, renal carcinoma, pancreatic cancer, epithelial carcinoma, gastric cancer, colon carcinoma, duodenal cancer, pancreatic adenocarcinoma, mesothelioma, glioblastoma multiforme, astrocytoma, multiple myeloma, prostate carcinoma, hepatocellular carcinoma, cholangiosarcoma, pancreatic adenocarcinoma, head and neck squamous cell carcinoma, colorectal cancer, intestinal-type gastric adenocarcinoma, cervical squamous-cell carcinoma, osteosarcoma, epithelial ovarian carcinoma, acute lymphoblastic lymphoma, myeloproliferative neoplasms, and sarcoma.

Cancer cells that can be treated by the methods of this disclosure can include cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; non-encapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhandomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcorna; hemangioendothelioma, malignant; Kaposi's sarcoma; hemangiopericytoma, malignant; lymphangiosarcoma; osteosarcoma; juxtacortical osteosarcoma; chondrosarcoma; chondrohlastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In some cases; solid cancers that are metastatic can be treated using the modified oncolytic viruses of this disclosure, such as a modified oncolytic poxvirus that is advantageous for systemic delivery. In some cases, solid cancers that are inaccessible or difficult to access, such as for purpose of intratumoral delivery of therapeutic agents, can be treated using a modified oncolytic poxvirus of this disclosure, that is advantageous for systemic delivery. Cancers that are associated with increased expression of free fatty acids can, in some examples, be treated using the modified oncolytic poxvirus of this disclosure that is advantageous for systemic delivery and forms increased amounts of EEV.

This disclosure also contemplates methods for inhibiting or preventing local invasiveness or metastasis, or both, of any type of primary cancer. For example, the primary cancer can be melanoma, non-small cell lung, small-cell lung, lung, hepatocarcinoma, retinoblastoma, astrocytoma, glioblastoma, gum, tongue, leukemia, neuroblastoma, head, neck, breast, pancreatic, prostate, renal, bone, testicular, ovarian, mesothelioma, cervical, gastrointestinal, lymphoma, brain, colon, or bladder. In certain embodiments, the primary cancer can be lung cancer. For example, the lung cancer can be non-small cell lung carcinoma. Moreover, this disclosure can be used to prevent cancer or to treat pre-cancers or premalignant cells, including metaplasias, dysplasias, and hyperplasias. It can also be used to inhibit undesirable but benign cells, such as squamous metaplasia, dysplasia, benign prostate hyperplasia cells, hyperplastic lesions, and the like. In some embodiments, the progression to cancer or to a more severe form of cancer can be halted, disrupted, or delayed by methods of this disclosure involving a modified oncolytic poxvirus as discussed herein.

Furthermore, a modified oncolytic poxvirus as disclosed herein can be administered for treatment of tumors with high bioavailability of free fatty acids in the tumor microenvironment. In some instances, free fatty acids released by adipocytes in tumors in obese patients can feed and enhance the replication of a modified oncolytic poxvirus within the tumor, and formation of FEV form of the virus. The advantage can also be realized in non-obese patients, especially patients who have peritoneal cancer. For example, several peritoneal cancers can be targets for therapy using the modified oncolytic viruses of this disclosure as these tend to grow in omentum wall and can be fed by adipocytes, and as mentioned above free fatty acids released by adipocytes in tumors can feed and enhance the replication of the modified oncolytic virus within the tumor. The modified oncolytic poxvirus as disclosed herein can form an increased titer of extracellular enveloped virus (EEV) in tumors with high bioavailability of free fatty acids.

In some embodiments is provided a method of treating a tumor by administering cells that are infected with a modified poxvirus as disclosed herein. The infected cells can be administered a subject, for instance intratumorally, whereby the modified poxvirus produces a population of viral particles in situ (such as within a tumor or a tumor microenvironment) containing a high percentage of EEV particles (e.g., at least about 10%, about 20%, about 30%, about 40%, about 50%, about 60%, about 70%, about 80%, about 90%, or greater). The EEV particles can then be harvested from the subject, such as from a biological sample isolated from the subject, and used for subsequent systemic delivery (e.g., intravenous delivery) to the subject. In some cases, this can enhance the systemic dissemination of the modified oncolytic virus within the subject and improve therapeutic outcome.

### Methods of Treatment and Assaying the Efficacy and Pharmacokinetics

This disclosure provides, in some embodiments, methods for treating a subject by administration of a modified oncolytic poxvirus as disclosed herein.

Provided is a method of producing a toxic effect in a cancer cell comprising administering to the cancer cell, a therapeutically effective amount of a modified oncolytic poxvirus, as described above, or a pharmaceutical composition containing the same. This disclosure further provides a method of inhibiting at least one of growth and proliferation of a second cancer cell comprising administering, to a first cancer cell, a modified oncolytic poxvirus as described above such that the first cancer cell is infected with said virus. Thus, in some embodiments of the methods disclosed here, it is contemplated that not every cancer or tumor cell is infected upon administering a therapeutically effective amount of a modified oncolytic poxvirus, as described herein, or a pharmaceutical composition containing the same, and growth of non-infected cells can be inhibited without direct infection.

In some examples, to induce oncolysis, kill cells, inhibit growth, inhibit metastases, decrease tumor size and otherwise reverse or reduce the malignant phenotype of tumor cells, using the methods and compositions of the present disclosure, a cancer cell or a tumor can be contacted with a therapeutically effective dose of an exemplary modified oncolytic poxvirus as described herein or a pharmaceutical composition containing the same. In certain embodiments, an effective amount of a modified oncolytic poxvirus of the present disclosure, or a pharmaceutical composition thereof, can include an amount sufficient to induce oncolysis, the disruption or lysis of a cancer cell or the inhibition or reduction in the growth or size of a cancer cell. Reducing the growth of a cancer cell may be manifested, for example, by cell death or a slower replication rate or reduced growth rate of a tumor comprising the cell or a prolonged survival of a subject containing the cancer cell.

Provided, in some embodiments, is a method of treating a subject having a cancer or a tumor comprising administering, to the subject, an effective amount of a modified virus, as described above. An effective amount in such method can include an amount that reduces growth rate or spread of the cancer or that prolongs survival in the subject. This disclosure provides a method of reducing the growth of a tumor, which method can comprise administering, to the tumor, an effective amount of a modified oncolytic poxvirus as described above. In certain embodiments, an effective amount of a modified oncolytic poxvirus, or a pharmaceutical composition thereof, can include an amount sufficient to induce the slowing, inhibition or reduction in the growth or size of a tumor and can include the eradication of the tumor. Reducing the growth of a tumor may be manifested, for example, by reduced growth rate or a prolonged survival of a subject containing the tumor.

This disclosure also provides a method of determining the infectivity or anti-tumor activity, or amount of tumor specific viral replication of a modified oncolytic poxvirus as described herein, which method can comprise; (i) administering to a subject a therapeutically effective amount of a modified oncolytic poxvirus or a pharmaceutical composition according to the present disclosure, which further expresses a luciferase reporter gene, alone or in combination with a further therapy; (ii) collecting a first biological sample from the subject immediately after administering the virus and determining the level of the luciferase reporter in the first biological sample (iii) collecting a second biological sample from the subject following the administration in step (ii) and (iii) detecting the level of the luciferase reporter in the second biological sample, wherein the modified oncolytic poxvirus is determined to be infective, demonstrate anti-tumor activity, exhibit tumor specific viral replication if the level of luciferase is higher in step (iii) than in step (ii). The second biological sample is collected about 30 mins, about 1 hour, about 2 hours, about 3 hours, about 4 hours, about 5 hours, about 6 hours, about 7 hours, about 8 hours, about 9 hours, about 10 hours, about 12 hours, about 15 hours, about 24 hours, about 36 hours, about 48 hours, about 72 hours, about 4 days, about 5 days, about 6 days, about 7 days, about 8 days, about 9 days, about 10 days, about 11 days, about 12 days, about 13 days, about 14 days, about 15 days, about 1 month, to about 2 months after the administration in step (i). In some embodiments, the method of mentioned above can further comprise, detecting in steps (i) and (iii), the level of one or more assaying cytokine levels, e.g., IL-2, IL-7, IL-8, IL-10, IFN-γ, GM-CSF, TNF-α, IL-6, IL-4, IL-5, and IL-13, in plasma samples collected from a subject after administering to said subject a therapeutically effective amount of a modified oncolytic poxvirus of the present disclosure, such as a modified oncolytic poxvirus as described herein or a pharmaceutical composition comprising the same. In some embodiments of this disclosure, the increase in luciferase bioluminescence between steps and (iv) mentioned above is higher for a modified oncolytic poxvirus as described herein, compared to that in an otherwise identical virus that does not comprise the modifications in the modified oncolytic poxvirus. Other exemplary techniques for detecting and monitoring viral load after administration of the modified oncolytic poxvirus es include real-time quantitative PCR.

Further provided is a method of monitoring the pharmacokinetics following administration of a therapeutically effective amount of modified oncolytic poxvirus according to the present disclosure or a pharmaceutical composition containing the poxvirus, as described herein. An exemplary method for monitoring the pharmacokinetics can comprise the following steps: (i) administering to the subject a therapeutically effective amount of a modified oncolytic poxvirus or a pharmaceutical composition comprising the same, alone or in combination with a further therapy; (ii) collecting biological samples from the subject at one or more time points selected from about 15 minutes, about 30 minutes, about 45 mins, about 60 mires, about 75 mins, about 90 mins, about 120 mins, about 180 mins, and about 240 rains, about 5 hours, about 6 hours, about 7 hours, about 8 hours, about 9 hours, about 10 hours, about 12 hours, about 15 hours, about 24 hours, about 36 hours, about 48 hours, about 72 hours, about 4 days, about 5 days, about 6 days, about 7 days, about 8 days, about 9 days, about 10 days, about 11 days, about 12 days, about 13 days, about 14 days, about 15 days, about 1 month, to about 2 months after the administration in step (i) and (iii) detecting the quantity of the viral genome (or a reporter gene inserted within the viral genome, such as luciferase) in the biological samples collected at the above mentioned time points. In some instances, viral genome copies/mt can be highest in the sample collected at the 15 mins time point and further the sample collected at the 240 mins time point may not contain a detectable quantity of the viral genome. Therefore, in some instances, a viral peak can be observed at about 15 mins following administration and majority of the viruses can be cleared from the subject's system after about 240 mins (or 4 hours). In some instances, a first viral peak can be observed after about 15 mins following administration and a second viral peak can be observed in the biological samples collected in the subsequent time points, e.g., at about 30 mins, about 45 mins, about 60 mins, or about 90 mins. The biological sample can be, in exemplar embodiments, blood, and the quantity of viral genome/mL can be determined by quantitative PCR or other appropriate techniques. In some examples, a first viral peak can be observed after about 15 mins following administration and a second viral peak can be observed after about 30 mins, about 1 hour, about 2 hours, about 3 hours, about 4 hours, about 5 hours, about 6 hours, about 7 hours, about 8 hours, about 9 hours, about 10 hours, about 12 hours, about 15 hours, about 24 hours, about 36 hours, about 48 hours, about 72 hours, about 4 days, about 5 days, about 6 days, about 7 days, about 8 days, about 9 days, about 10 days, about 11 days, about 12 days, about 13 days, about 14 days, about 15 days, about 1 month, to about 2 months following administration of a modified oncolytic virus of the present disclosure, such as an oncolytic poxvirus as described herein.

In some instances, tumor-selective replication of a modified oncolytic poxvirus can be measured through use of a reporter gene, such as a luciferase gene. In some embodiments, the luciferase gene can be inserted into the genome of a virus, and a tumor cell can be infected with the virus. Bioluminescence in infected tumor cells can be measured to monitor tumor-selective replication. Some examples show an increase in luciferase reporter bioluminescence in a modified oncolytic poxvirus of this disclosure, compared to that in an otherwise identical oncolytic poxvirus that does not contain the modifications in the modified oncolytic virus.

### Delivery of Modified Oneolytic Viruses

In some embodiments, amount of a modified oncolytic poxvirus of this disclosure administered to a subject can be between about 10³ and 10¹² infectious viral particles or plaque forming units (PRY), or between about 10⁵ and 10¹⁰ PFU, or between about 10⁵ and 10⁸ PFU, or between about 10⁸ and 10¹⁰ PFU. In some embodiments, the amount of a modified oncolytic poxvirus of this disclosure administered to a subject can be between about 10³ and 10¹² viral particles or plaque forming units (PFU), or between about 10⁵ and 10¹⁰ PFU, or between about 10⁵ and 10⁸ PFU, or between about 10⁸ and 10¹⁰ PFU, In some embodiments, a modified oncolytic poxvirus of this disclosure can be administered at a dose that can comprise about 10³ PFU/dose to about 10⁴ PFU/dose, about 10⁴ PFU/dose to about 10⁵ PFU/dose, about 10⁵ PFU/dose to about 10⁶ PFU/dose, about 10⁷ PFU/dose to about 10⁸ PFU/dose, about 10⁹ PFU/dose to about 10¹⁰ PFU/dose, about 10¹⁰ PFU/dose to about 10¹¹ PFU/dose, about 10¹¹ PFU/dose to about 10¹² PFU/dose, about 10¹² PFU; to about 10¹³ PFU/dose, about 10¹³ PFU/dose to about 10¹⁴ PFU/dose, or about 10¹⁴ PFU/dose to about 10¹⁵ PFU/dose. In some embodiments, a modified oncolytic poxvirus of this disclosure can be administered at a dose that can comprise about 2×10³ PFU/dose, 3×10³ PFU/dose, 4×10³ PFU/dose, 5×10³ PFU/dose, 6×10³ PFU/dose, 7×10³ PFU/dose, 8×10³ PFU/dose, 9×10³ PFU/dose, about 10⁴ PFU/dose, about 2×10⁴ PFU/dose, about 3×10⁴ PFU/dose, about 4×10⁴ PFU/dose, about 5×10⁴ PFU/dose, about 6×10⁴ PFU/dose, about 7×10⁴ PFU/dose, about 8×10⁴ PFU/dose, about 9×10⁴ PFU/dose, about 10⁵ PFU/dose, 2×10⁵ PFU/dose, 3×10³ PFU/dose, 4×10⁵ PFU/dose, 5×10⁵ PFU/dose, 6×10⁵ PFU/dose, 7×10⁵ PFU/dose, 8×10⁵ PFU/dose, 9×10⁵ PFU/dose, about 10⁶ PFU/dose, about 2×10⁶ PFU/dose, about 3×10⁶ PFU/dose, about 4×10⁶ PFU/dose, about 5×10⁶ PFU/dose, about 6×10⁶ PFU/dose, about 7×10⁶ PFU/dose, about 8×10⁶ PFU/dose, about 9×10⁶ PFU/dose, about 10⁷ PFU/dose, about 2×10⁷ PFU/dose, about 3×10⁷ PFU/dose, about 4×10⁷ PFU/dose, about 5×10⁷ PFU/dose, about 6×10⁷ PFU/dose, about 7×10⁷ PFU/dose, about 8×10⁷ PFU/dose, about 9×10⁷ PFU/dose, about 10⁸ PFU/dose, about 2×10⁸ PFU/dose, about 3×10⁸ PFU/dose, about 4×10⁸ PFU/dose, about 5×10⁸ PFU/dose, about 6×10⁸ PFU/dose about 7×10⁸ PFU/dose, about 8×10⁸ PFU/dose, about 9×10⁸ PFU/dose, about 10⁹ PFU/dose, about 2×10⁹ PFU/dose, about 3×10⁹ PFU/dose, about 4×10⁹ PFU/dose, about 5×10⁹ PFU/dose, about 6×10⁹ PFU/dose, about 7×10⁹ PFU/dose, about 8×10⁹ PFU/dose, about 9×10⁹ PFU/dose, about 10¹⁰ PFU/dose, about 2×10¹⁰ PFU/dose, about 3×10¹⁰ PFU/dose, about 4×10¹⁰ PFU/dose, about 5×10¹⁰ PFU/dose, about 6×10¹⁰ PFU/dose, about 7×10¹⁰ PFU/dose, about 8×10¹⁰ PFU/dose, about 9×10¹⁰ PFU/dose, about 10¹⁰ PFU/dose, about 2×10¹⁰ PFU/dose, about 3×10¹⁰ PFU/dose, about 4×10¹⁰ PFU/dose, about 5×10¹⁰ PFU/dose, about 6×10¹⁰ PFU/dose, about 7×10¹⁰ PFU/dose, about 8×10¹⁰ PFU/dose, about 9×10¹⁰ PFU/dose, about 10¹⁰ PFU/dose, about 2×10¹¹ PFU/dose, about 3×10¹¹ PFU/dose, about 4×10¹¹ PFU/dose about 5×10¹¹ PFU/dose, about 6×10¹¹ PFU/dose about 7×10¹¹ PFU/dose, about 8×10¹¹ PFU/dose about 9×10¹¹ PFU/dose, or about 10¹² PFU/dose, about 10¹² PFU/dose to about 10¹³ PFU/dose, about 10¹³ PFU/dose to about 10¹⁴ PFU/dose, or about 10¹⁴ PFU/dose to about 10¹⁵ PFU/dose. In some embodiments, a modified oncolytic poxvirus of this disclosure can be administered at a dose that can comprise 5×10⁹ PFU/dose. In some embodiments, a modified oncolytic poxvirus of this disclosure can be administered at a dose that can comprise up to 5×10⁹ PFU/dose.

In some embodiments, a modified oncolytic poxvirus of this disclosure, can be administered at a dose that can comprise about 10³ viral particles/dose to about 10⁴ viral particles/dose, about 10⁴ viral particles/dose to about 1.0⁵ viral particles/dose, about 10⁵ viral particles/dose to about 10⁶ viral particles/dose, about 10⁷ viral particles/dose to about 10⁸ viral particles/dose, about 10⁹ viral particles/dose to about 10¹⁰ viral particles/dose, about 10¹⁰ viral particles idose to about 10¹¹ viral particles/dose, about 10¹¹ viral particles/dose to about 10¹² viral particles/dose, about 10¹² viral particles/dose to about 10¹³ viral particles/dose, about 10¹³ viral particles/dose to about 10¹⁴ viral particles/dose, or about 10¹⁴ viral particles/dose to about 10¹⁵ viral particles/dose,

In some embodiments, a modified oncolytic, poxvirus of this disclosure can be administered at a dose that can comprise about 10³ PFU/kg to about 10⁴ PFU/kg, about 10⁴ PFU/kg to about 10⁵ PFU/kg, about 10⁵ PFU/kg to about 10⁶ PFU/kg, about 10⁷ PFU/kg to about 10⁸ PFU/kg, about 10⁹ PFU/kg to about 10¹⁰ PFU/kg, about 10¹⁰ PFU/kg to about 10¹¹ PFU/kg, about 10¹¹ PFU/kg to about 10¹² PFU/kg, about 10¹² PFU/kg to about 10¹³ PFU/kg, about 10¹³ PFU/kg to about 10¹⁴ PFU/kg, or about 10¹⁴ PFU/kg to about 10¹⁵ PFU/kg. In some embodiments, a modified oncolytic poxvirus of this disclosure can be administered at a dose that can comprise about 2×10³ PFU/kg, 3×10³ PFU/kg 4×10³ PFU/kg, 5×10³ PFU/kg, 6×10³ PFU/kg, 7×10³ PFU/kg, 8×10³ PFU/kg, 9×10³ PFU/kg about 10⁴ PFU/kg, about 2×10⁴ PFU/kg, about 3×10⁴ PFU/kg, about 4×10⁴ PFU/kg, about 5×10⁴ PFU/kg, about 6×10⁴ PFU/kg, about 7×10⁴ PFU/kg, about 8×10⁴ PFU/kg, about 9×10⁴ PFU/kg, about 10⁵ PFU/kg, 2×10⁵ PFU/kg, 3×10⁵⁻PFU/kg 4×10⁵ PFU/kg, 5×10⁵ PFU/kg, 6×10⁵ PFU/kg 7×10⁵ PFU/kg, 8×10⁵ PFU/kg, 9×10⁵ PFU/kg, about 10⁶ PFU/kg, about 2×10⁶ PFU/kg, about 3×10⁶ PFU/kg, about 4×10⁶ PFU/kg, about 5×10⁶ PFU/kg, about 6×10⁶ PFU/kg, about 7×10⁶ PFU/kg, about 8×10⁶ PFU/kg, about 9×10⁶ PFU/kg, about 10⁷ PFU/kg, about 2×10⁷ PFU/kg, about 3×10⁷ PFU/kg, about 4×10⁷ PFU/kg, about 5×10⁷ PFU/kg, about 6×10⁷ PFU/kg, about 7×10⁷ PFU/kg, about 8×10⁷ PFU/kg, about 9×10⁷ PFU/kg, about 10⁸ PFU/kg, about 2×10⁸ PFU/kg, about 3×10⁸ PFU/kg, about 4×10⁸ PFU/kg, about 5×10⁸ PFU/kg, about 6×10⁸ PFU/kg, about 7×10⁸ PFU/kg, about 8×10⁸ PFU/kg, about 9×10⁸ PFU/kg, about 10⁹ PFU/kg, about 2×10⁹ PFU/kg, about 3×10⁹ PFU/kg, about 4×10⁹ PFU/kg, about 5×10¹⁰ PFU/kg, about 6×10¹⁰ PFU/kg, about 7×10⁹ PFU/kg, about 8×10⁹ PFU/kg, about 9×10⁹ PFU/kg, about 10¹⁰ PFU/kg, about 2×10¹⁰ PFU/kg, about 3×10¹⁰ PFU/kg, about 4×10¹⁰ PFU/kg, about 5 xl 0′° PFU/kg, about 6×10¹⁰ PFU/kg, about 7×10¹⁰ PFU/kg about 8×10¹⁰ PFU/kg, about 9×10¹⁰ PFU/kg, about 10¹⁰ PFU/kg, about 2×10¹⁰ PFU/kg, about 3×10¹⁰ PFU/kg, about 4×10¹⁰ PFU/kg, about 5×10¹⁰ PFU/kg, about 6×10¹⁰ PFU/kg, about 7×10¹⁰ PFU/kg about 8×10¹⁰ PFU/kg, about 9×10¹⁰ PFU/kg, about 10¹¹ PFU/kg, about 2×10¹¹ PFU/kg about 3×10¹¹ PFU/kg, about 4×10¹¹ PFU/kg, about 5×10¹¹ PFU/kg about 6×10¹¹ PFU/kg, about 7×10¹¹ PFU/kg, about 8×10¹¹ PFU/kg, about 9×10¹¹ PFU/kg, or about 10¹² PFU/kg, about 10¹² PFU/kg to about 10¹³ PFU/kg, about 10¹³ PFU/kg to about 10¹⁴ PFU/kg, or about 10¹⁴ PFU/kg to about 10¹⁵ PFU/kg. In some embodiments, a modified oncolytic poxvirus of this disclosure can be administered at a dose that can comprise 5×10⁹ PFU/kg. In some embodiments, a modified oncolytic pox virus of this disclosure can be administered at a dose that can comprise up to 5×10⁹ PFU/kg.

In some embodiments, a modified oncolytic poxvirus of this disclosure can be administered at a dose that can comprise about 10 viral particles/kg to about 10⁴ viral particles/kg, about 10⁴ viral particles/kg to about 10⁵ viral particles/kg, about 10⁵ viral particles/kg to about 10⁶ viral particles/kg, about 10⁷ viral particles/kg to about 10⁸ viral particles/kg, about 10⁹ viral particles/kg to about 10¹⁰ viral particles/kg, about 10¹⁰ viral particles/kg to about 10¹¹ viral particles/kg, about 10¹¹ viral particles/kg to about 10¹² viral particles/kg, about 10¹² viral particles/kg to about 10¹³ viral particles/kg, about 10¹³ viral particles/kg to about 10¹⁴ viral particles/kg, or about 10¹⁴ viral particles/kg to about 10¹⁵ viral particles/kg.

A liquid dosage form of a modified oncolytic poxvirus as described herein can comprise, in certain embodiments, a viral dose of about 10³ PFU/mL: to about 10⁴ PFU/mL, about 10′¹ PFU/mL to about 10⁵ PFU/mL, about 10⁵ PFU/mL to about 10⁶ PFU/mL, about 10⁷ PFU/ML, to about 10⁸ PFU/mL, about 10⁹ PFU/mL to about 10¹⁰ PFU/mL, about 10¹⁰ PFU/mL to about 10¹¹ PFU/mL, about 10¹¹ PFU/mL to about 10¹² PFU/mL about 10¹² PFU/mL to about 10¹³ PFU/mL, about 10¹³ PFU/mL to about 10¹⁴ PFU/mL, or about 10¹⁴ PFU/mL to about 10¹⁵ PFU/mL. In some embodiments, a modified oncolytic poxvirus of this disclosure can be administered at a dose that can comprise about 2×10³ PFU/mL, 3×10³ PFU/mL, 4×10³ PFU/mL, 5×10³ PFU/mL, 6×10³ PFU/mL, 7×10³ PFU/mL 8×10³ PFU/mL, 9×10³ PFU/mL about 10⁴ PFU/mL about 2×10⁴ PFU/mL about 3×10⁴ PFU/mL, about 4×10⁴ PFU/mL, about 5×10⁴ PFU/mL, about 6×10⁴ PFU/ML, about 7×10⁴ PFU/mL, about 8×10⁴ PFU/mL, about 9×10⁴ PFU/mL about 10⁵ PFU/mL, 2×10⁵ PFU/mL, 3×10⁵ PFU/mil, 4×10⁵ PFU/mL 5×10⁵ PFU/mL, 6×10⁵ PFU/mL, 7×10⁵ PFU/mL, 8×10⁵ PFU/mL, 9×10⁵ PFU/mL, about 10⁶ PFU/mL, about 2×10⁶ PFU/mL, about 3×10⁶ PFU/mL, about 4×10⁶ PFU/mL, about 5×10⁶ PFU/mL, about 6×10⁶ PFU/mL, about 7×10⁶ PFU/mL, about 8×10⁶ PFU/mL, about 9×10⁶ PFU/mL, about 10⁷ PFU/mL about 2×10⁷ PFU/mL, about 3×10⁷ PFU/mL, about 4×10⁷ PFU/mL, about 5×10⁷ PFU/mL, about 6×10⁷ PFU/mL, about 7×10⁷ PFU/mL, about 8×10⁷ PFU/mL, about 9×10⁷ PFU/mL, about 10⁸ PFU/mL, about 2×10⁸ PFU/mL, about 3×10⁸ PFU/mL, about 4×10⁸ PFU/mL, about 5×10⁸ PFU/mL, about 6×10⁸ PFU/mL, about 7×10⁸ PFU/mL, about 8×10⁸ PFU/mL, about 9×10⁸ PFU/mL, about 10⁹ PFU/mL, about 2×10⁹ PFU/mL, about 3×10⁹ PFU/mL, about 4×10⁹ PFU/mL, about 5×10⁹ PFU/mL, about 6×10⁹ PFU/mL, about 7×10⁹ PFU/mL, about 8×10⁹ PFU/mL, about 9×10⁹ PFU/mL, about 10¹⁰ PFU/mL, about 2×10¹⁰ PFU/mL, about 3×10¹⁰ PFU/mL, about 4×10¹⁰ PFU/mL, about 5×10¹⁰ PFU/mL, about 6×10¹⁰ PFU/mL, about 7×10¹⁰ PFU/mL, about 8×10¹⁰ PFU/mL, about 9×10¹⁰ PFU/mL, about 10¹⁰ PFU/mL, about 2×10¹⁰ PFU/mL, about 3×10¹⁰ PFU/mL, about 4×10¹⁰ PFU/mL, about 5×10¹⁰ PFU/mL, about 6×01¹⁰ PFU/mL, about 7×10¹⁰ PFU/mL, about 8×10¹⁰ PFU/mL, about 9×10¹⁰ PFU/mL, about 10¹¹ PFU/mL, about 2×10¹¹ PFU/mL, about 3×10¹¹ PFU/mL, about 4×10¹¹ PFU/mL, about 5×10¹³ PFU/mL, about 6×10¹¹ PFU/mL, about 7×10¹¹ PFU/mL, about 8×10¹³ PFU/mL, about 9×10¹³ PFU/mL, or about 10¹² PFU/mL, about 10¹² PFU/mL to about 10¹³ PFU/mL, about 10¹³ PFU/mL to about 10¹⁴ PFU/mL, or about 10¹⁴ PFU/mL to about 10¹⁵ PFU/mL. In some embodiments, a modified oncolytic poxvirus of this disclosure can be administered at a dose that can comprise 5×10⁹ PFU/mL. In some embodiments, a modified oncolytic poxvirus of this disclosure can be administered at a dose that can comprise up to 5×10⁹ PFU/mL.

In some instances, where the modified oncolytic poxvirus can be administered by an injection, the dosage can comprise about 10¹ viral particles per injection. 10⁴ viral particles per injection, 10⁵ viral particles per injection, 10⁶ viral particles per injection, 10⁷ viral particles per injection, 10⁸ viral particles per injection, 10⁹ viral particles per injection, 10¹⁰ viral particles per injection, 10¹¹ viral particles per injection, 10¹² viral particles per injection, 2×10¹² viral particles per injection, 10¹³ viral particles per injection, 10¹⁴ viral particles per injection, or 10¹⁵ viral particles per injection. In further instances, where the modified oncolytic poxvirus is administered by an injection, the dosage can comprise about 10¹ infectious viral particles per injection, 10⁴ infectious viral particles per injection, 10⁵ infectious viral particles per injection, 10⁶ infectious viral particles per injection, 10⁷ infectious viral particles per injection, 10⁸ infectious viral particles per injection, 10⁹ infectious viral particles per injection, 10¹⁰ infectious viral particles per injection, 10¹¹ infectious viral particles per injection, 10¹² infectious viral particles per injection, 2×10¹² infectious viral particles per injection, 10¹³ infectious viral particles per injection, 10¹⁴ infectious viral particles per injection, or 10¹⁵ infectious viral particles per injection. In additional embodiments, a modified oncolytic poxvirus of this disclosure can be administered at a dose that can be about 10³ Tissue Culture Inhibitor Dose 50% (TCID₅₀)/kg, 10⁴ TCID₅₀/kg, 10⁴ TCID₅₀/kg, 10⁴ TCID₅₀/kg, 10⁴ TCID₅₀/kg, 10⁴ TCID₅₀/kg, 10⁴ TCID₅₀/kg, 10⁴ TCID₅₀/kg, 10⁴ TCID₅₀/kg, 10⁴ TCID₅₀/kg, 10⁴ TCID₅₀/kg, 10⁴ TCID₅₀/kg, 10⁴ TCID₅₀/kg, 3×10⁸ TCID₅₀1 kg, 4×10⁸ TCID₅₀/kg, 5×10⁸ TCID₅₀/kg, 3×10⁹ TCID₅₀/kg, 4×10⁹ TCID₅₀/kg, 5×10⁹ TCID₅₀/kg, 3×10¹⁰ TCID₅₀, 4×10¹⁰ TCID₅₀/kg, or 4×10¹⁰ TCID₅₀/kg. Note that herein 10^{x} is alternatively expressed as 1 eX. In certain embodiments, the modified oncolytic poxvirus can be administered in one or more doses. In certain embodiments, the virus can be administered in an amount sufficient to induce oncolysis in at least about 20% of cells in a tumor, in at least about 30% of cells in a tumor, in at least about 40% of cells in a tumor, in at least about 50% of cells in a tumor, in at least about 60% of cells in a tumor, in at least about 70% of cells in a tumor, in at least about 80% of cells in a tumor, or in at least about 90% of cells in a tumor. In certain embodiments, a single dose of virus can refer to the amount administered to a subject or a tumor over a. 1, 2, 5, 10, 15, 20 or 24 hour period. In certain embodiments, the dose can be spread over time or by separate injection. In certain embodiments, multiple doses (e.g., 2, 3, 4, 5, 6 or more doses) of the poxvirus can be administered to the subject, for example, where a second treatment can occur within 1, 2, 3, 4, 5, 6, 7 days or weeks of a first treatment. In certain embodiments, multiple doses of the modified oncolytic v can be administered to the subject over a period of 1, 2, 3, 4, 5, 6, 7 or more days or weeks. In certain embodiments, the oncolytic vaccina virus or the pharmaceutical composition as described herein can be administered over a period of about 1 week to about 2 weeks, about 2 weeks to about 3 weeks, about 3 weeks to about 4 weeks, about 4 weeks to about 5 weeks, about 6 weeks to about 7 weeks, about 7 weeks to about 8 weeks, about 8 weeks to about 9 weeks, about 9 weeks to about 10 weeks, about 10 weeks to about 11 weeks, about 11 weeks to about 12 weeks, about 12 weeks to about 24 weeks, about 24 weeks to about 48 weeks, about 48 weeks or about 52 weeks, or longer. The frequency of administration of the oncolytic poxvirus or the pharmaceutical composition as described herein can be, in certain instances, once daily, twice daily, once every week, once every three weeks, once every four weeks (or once a month), once every 8 weeks (or once every 2 months), once every 12 weeks (or once every 3 months), or once every 24 weeks (once every 6 months). In some embodiments of the methods disclosed herein, the oncolytic poxvirus or the pharmaceutical composition can be administered, independently, in an initial dose for a first period of time, an intermediate dose fora second period of time, and a high dose for a third period of time. In some embodiments, the initial dose can be lower than the intermediate dose and the intermediate dose can be lower than the high dose. In some embodiments, the first, second, and third periods of time can be, independently, about 1 week to about 2 weeks, about 2 weeks to about 3 weeks, about 3 weeks to about 4 weeks, about 4 weeks to about 5 weeks, about 6 weeks to about 7 weeks, about 7 weeks to about 8 weeks, about 8 weeks to about 9 weeks, about 9 weeks to about 10 weeks, about 10 weeks to about 11 weeks, about 11 weeks to about 12 weeks, about 12 weeks to about 24 weeks, about 24 weeks to about 48 weeks, about 48 weeks or about 52 weeks, or longer.

In some examples, the subject can be put on a reduced carbohydrate diet, e.g., a ketogenic diet prior to, concurrent with, and following administration of the modified oncolytic poxvirus es or the pharmaceutical composition comprising the same, as described herein, according to any of the methods of treatment described herein. In certain embodiments, the subject can be put on a diet that can comprise consuming less than 500 grams of carbohydrates per day, less than 450 grams of carbohydrates per day, less than 450 grams of carbohydrates per day, less than 400 grams of carbohydrates per day, less than 350 grams of carbohydrates per day, less than 300 grams of carbohydrates per day, less than 250 grams of carbohydrates per day, less than 200 grains of carbohydrates per day, less than 150 grams of carbohydrates per day, less than 100 grams of carbohydrates per day, less than 90 grams of carbohydrates per day, less than 80 grams of carbohydrates per day, less than 70 grams of carbohydrates per day, less than 60 grams of carbohydrates per day, less than 50 grams of carbohydrates per day, less than 40 grams of carbohydrates per day, less than 30 grams of carbohydrates per day, less than 20 grams of carbohydrates per day, less or than 10 grams of carbohydrates per day.

An exemplary method for the delivery of a modified oncolytic poxvirus of the present disclosure or a pharmaceutical composition comprising the same, to cancer or tumor cells can be via intratumoral injection. However, alternate methods of administration can also be used, e.g., intravenous, via infusion, parenteral, intravenous, intradermal, intramuscular, transdermal, rectal, intraurethral, intravaginal, intranasal, intrathecal, or intraperitoneal. The routes of administration can vary with the location and nature of the tumor. In certain embodiments, the route of administration can be intradental, transdermal, parenteral, intravenous, intramuscular, intranasal, subcutaneous, regional (e.g., in the proximity of a tumor, particularly with the vasculature or adjacent vasculature of a tumor), percutaneous, intrathecal, intratracheal, intraperitoneal, intraarterial, intravesical, intratumoral, inhalation, perfusion, by lavage or orally, An injectable dose of the modified oncolytic poxvirus can be administered as a bolus injection or as a slow infusion. In certain embodiments, the modified oncolytic poxvirus can be administered to the patient from a source implanted in the patient. In certain embodiments, administration of the modified oncolytic poxvirus can occur by continuous infusion over a selected period of time. In some instances, an oncolytic poxvirus as described herein, or a pharmaceutical composition containing the same can be administered at a therapeutically effective dose by infusion over a period of about 15 mins, about 30 mins, about 45 rains, about 50 mins, about 55 mins, about 60 minutes, about 75 mins, about 90 mins, about 100 mins, or about 120 mins or longer. The modified oncolytic poxvirus or the pharmaceutical composition of the present disclosure can be administered as a liquid dosage, wherein the total volume of administration is about 1 mL to about 5 mL, about 5 mL to 10 mL, about 15 mL to about 20 mL, about 25 mL to about 30 mL, about 30 mL to about 50 mL about 50 mL to about 100 mL, about 100 mL to 150 mL, about 150 mL to about 200 mL, about 200 nit to about 250 mL, about 250 mL to about 300 mL, about 300 mL to about 350 mL, about 350 mL to about 400 mL, about 400 mL to about 450 mL, about 450 mL, to 500 mL, about 500 mL to 750 mL, or about 750 mL to 1000 mL,

### Pharmaceutical Compositions

Pharmaceutical compositions containing a modified oncolytic poxvirus, as described herein, can be prepared as solutions, dispersions in glycerol, liquid polyethylene glycols, and any combinations thereof in oils, in solid dosage forms, as inhalable dosage forms, as intranasal dosage forms, as liposomal formulations, dosage forms comprising nanoparticles, dosage forms comprising microparticles, polymeric dosage forms, or any combinations thereof. In some embodiments, a pharmaceutical composition as described herein can comprise a stabilizer and a buffer. In some embodiments, a pharmaceutical composition as described herein can comprise a solubilizer, such as sterile water, Tris-buffer. In some embodiments, a pharmaceutical composition as described herein can comprise an excipient. An excipient can be an excipient described in the Handbook of Pharmaceutical Excipients, American Pharmaceutical Association (1986). Non-limiting examples of suitable excipients can include a buffering agent, a preservative, a stabilizer, a binder, a compaction agent, a lubricant, a chelator, a dispersion enhancer, a disintegration agent, a flavoring agent, a sweetener, a coloring agent.

In some embodiments an excipient can be a buffering agent. Non-limiting examples of suitable buffering agents can include sodium citrate, magnesium carbonate, magnesium bicarbonate, calcium carbonate, and calcium bicarbonate. As a buffering agent, sodium bicarbonate, potassium bicarbonate, magnesium hydroxide, magnesium lactate, magnesium gluconate, aluminium hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts or combinations thereof can be used in a pharmaceutical formulation.

In some embodiments an excipient can comprise a presentative. Non-limiting examples of suitable preservatives can include antioxidants, such as alpha-tocopherol and ascorbate, and antimicrobials, such as parabens, chlorobutanol, and phenol. Antioxidants can further include but not limited to EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some instances a preservatives can include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitor.

In some embodiments a pharmaceutical composition as described herein can comprise a binder as an excipient. Non-limiting examples of suitable binders can include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof. The binders that can be used in a pharmaceutical formulation can be selected from starches such as potato starch, corn starch, wheat starch; sugars such as sucrose, glucose, dextrose, lactose, maltodextrin; natural and synthetic gums; gelatine; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol and water or a combination thereof.

In some embodiments a pharmaceutical composition as described herein can comprise a lubricant as an excipient. Non-limiting examples of suitable lubricants can include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil. The lubricants that can be used in a pharmaceutical formulation can be selected from metallic stearates (such as magnesium stearate, calcium stearate, aluminium stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate and talc or a combination thereof.

In some embodiments a pharmaceutical formulation can comprise a dispersion enhancer as an excipient. Non-limiting examples of suitable dispersants can include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments a pharmaceutical composition as described herein can comprise a disintegrant as an excipient. In some embodiments a disintegrant can be a non-effervescent disintegrant. Non-limiting examples of suitable non-effervescent disintegrants can include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. In some embodiments a disintegrant can be an effervescent disintegrant. Non-limiting examples of suitable effervescent disintegrants can include sodium bicarbonate in combination with citric acid, and sodium bicarbonate in combination with tartaric acid.

In some embodiments an excipient can comprise a flavoring agent. Flavoring agents incorporated into an outer layer can be chosen from synthetic flavor oils and flavoring aromatics; natural oils; extracts from plants, leaves, flowers, and fruits; and combinations thereof. In some embodiments a flavoring agent can be selected from the group consisting of cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiments an excipient can comprise a sweetener. Non-limiting examples of suitable sweeteners can include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin, Stevia Rebaudiana (Stevioside); chloro derivatives of sucrose such as sucralose; and sugar alcohols such as sorbitol, mannitol, xylitol, and the like.

In some instances, a pharmaceutical composition as described herein can comprise a coloring agent. Non-limiting examples of suitable color agents can include food, drug and cosmetic colors (FD&C), drug and cosmetic colors (D&C), and external drug and cosmetic colors (Ext. D&C). A coloring agents can be used as dyes or their corresponding lakes.

In some instances, a pharmaceutical composition as described herein can comprise a chelator. In some cases, a chelator can be a fungicidal chelator. Examples can include, but are not limited to: ethylenediamine-N,N,N′,N′-tetraacetic acid (EDTA); a disodium, trisodium, tetrasodium, dipotassium, tripotassium, dilithium and diammonium salt of EDTA; a barium, calcium, cobalt, copper, dysprosium, europium, iron, indium, lanthanum, magnesium, manganese, nickel, samarium, strontium, or zinc chelate of EDTA, trans-1,2-diaminocyclohexane-N,N,N′,N′-tetraaceticacid monohydrate; N,N-bis(2-hydroxyethyl)glycine; 1,3-diamino-2-hydroxypropane-N,N,N′,N′-tetritacetic acid; 1,3-diaminopropane-N,N,N′,N′-tetraacetic acid; ethylenediamine-N,N′-diacetic acid; ethylenediamine-N,N′-dipropionic acid dihydrochloride; ethylenediamine-N,N-bis(inethylenephosphonic acid) hemihydrate; N-(2-hydroxyethyl)ethylenediamine-N,N′,N′-triacetic acid; ethyleriediamine-N,N,N′,N′-tetrakis(methylenephosponic acid); O,O′-bis(2-aminoethyl)ethyleneglycol-N,N,N′,N′-tetraacetic acid; N,N-bis(2-hydroxybenzethylenediamine-N,N-diacetic acid; 1,6-hexamethylenediamine-N,N,N′,N′-tetraacetic acid; N-(2-hydroxyethyl)iminodi acetic acid; iminodiacetic acid; 1,2-diaminopropane-N,N,N′,N′-tetraacetic acid; nitrilotriacetic acid; nitrilotripropionic acid; the trisodium salt of nitrilotris(methylenephosphoric acid); 7,19,30-trioxa-1,4,10,13,16,22,27,33-octaazabicyclo[11,11,11]pentatriacontane hexahydrobromide; or triethylenetetramine-N,N,N′,N″,N′″,N′″-hexaacetic acid.

Also contemplated are combination products that include one or more modified oncolytic viruses disclosed herein and one or more other antimicrobial or antifungal agents, for example, polyenes such as amphotericin B, amphotericin B lipid complex (ABCD), liposomal amphotericin B (LAMB), and liposomal nystatin, azoles and triazoles such as voriconazole, fluconazole, ketoconazole, itraconazole, pozaconazole and the like; glucan synthase inhibitors such as caspoftmin, micafungin (FK463), and V-echinocandin (LY303366); griseofulvin; allylamines such as terbinafine; flucytosine or other antifungal agents, including those described herein. In addition, it is contemplated that a peptide can be combined with topical antifungal agents such as ciclopirox haloprogin, tolnaftate, undecylenate, topical nystatin, amorolfine, butenafine, naftifine, terbinafine, and other topical agents. In some instances, a pharmaceutical composition can comprise an additional agent. In some cases, an additional agent can be present in a therapeutically effective amount in a pharmaceutical composition.

Under ordinary conditions of storage and use, the pharmaceutical compositions as described herein can comprise a preservative to prevent the growth of microorganisms. In certain examples, the pharmaceutical compositions as described herein may not comprise a preservative. The pharmaceutical forms suitable for injectable use can include sterile aqueous solutions or dispersions and sterile powders for the extemporaneous preparation of sterile injectable solutions or dispersions. The pharmaceutical compositions can comprise a carrier which is a solvent or a dispersion medium containing, for example, water, ethanol, polyol (e.g., glycerol, propylene glycol, and liquid polyethylene glycol, and the like), and/or vegetable oils, or any combinations thereof. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the liquid dosage form can be suitably buffered if necessary and the liquid diluent rendered isotonic with sufficient saline or glucose. The liquid dosage forms are especially suitable for intravenous, intramuscular, subcutaneous, intratumoral, and intraperitoneal administration. In this connection, sterile aqueous media that can be employed will be known to those of skill in the art in light of the present disclosure. For example, one dosage may be dissolved in 1 mL to 20 mL of isotonic NaCl solution and either added to 100 mL to 1000 mL of a fluid, e.g., sodium-bicarbonate buffered saline, or injected at the proposed site of infusion.

In certain embodiments, sterile injectable solutions can be prepared by incorporating a modified onncolytic poxvirus as described herein or a pharmaceutical composition containing the same, in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, the pharmaceutical compositions can be administered in a manner compatible with the dosage formulation and in such amount as is therapeutically effective.

In certain embodiments, a pharmaceutical composition of this disclosure can comprise an effective amount of a modified oncolytic poxvirus, disclosed herein, combined with a pharmaceutically acceptable carrier. “Pharmaceutically acceptable,” as used herein, includes any carrier which does not interfere with the effectiveness of the biological activity of the active ingredients and/or that is not toxic to the patient to whom it is administered. Non-limiting examples of suitable pharmaceutical carriers can include phosphate buffered saline solutions, water, emulsions, such as oil/water emulsions, various types of wetting agents and sterile solutions. Additional non-limiting examples of pharmaceutically compatible carriers can include gels, bioadsorbable matrix materials, implantation elements containing the modified oncolytic virus or any other suitable vehicle, delivery or dispensing means or material. Such carriers can be formulated by conventional methods and can be administered to the subject at an effective amount.

### Methods of Production

The modified oncolytic poxviruses of this disclosure can be produced by methods known to one of skill in the art. In certain embodiments, the modified oncolytic virus can be propagated in suitable host cells, e.g., HeLa cells, 293 cells, or Vero cells, isolated from host cells and stored in conditions that promote stability and integrity of the virus, such that loss of infectivity over time is minimized. In some instances, the modified oncolytic virus, producing a high percentage of EEV particles can act as an improved inoculum in a process for manufacture of an oncolytic virus. In certain exemplary methods, the modified oncolytic poxviruses are propagated in host cells using cell stacks, roller bottles, or perfusion bioreactors. In some examples, downstream methods for purification of the modified oncolytic viruses can comprise filtration (e.g., depth filtration, tangential flow filtration, or a combination thereof), ultracentrifugation, or chromatographic capture. The modified oncolytic virus can be stored, e.g., by freezing or drying, such as by lyophilization. In certain embodiments, prior to administration, the stored modified oncolytic poxvirus can be reconstituted (if dried for storage) and diluted in a pharmaceutically acceptable carrier for administration.

Some embodiments provide that the modified oncolytic poxvirus as described herein, exhibit a higher titer in HeLa cells and 293 cells compared to an otherwise identical virus that does not comprise the modifications in the modified oncolytic virus. In certain instances, a higher titer in HeLa cells and 293 cells is seen in modified oncolytic poxvirus.

### Combination Therapies

In certain embodiments, the methods of this disclosure comprise administering a modified oncolytic poxvirus as disclosed herein or a pharmaceutical composition containing the same, followed by, and preceded by or in combination with one or more further therapy. Examples of the further therapy can include, but are not limited to, chemotherapy, radiation, oncolytic viral therapy with an additional virus, treatment with immunomodulatory proteins, an anti-cancer agent, or any combinations thereof. The further therapy can be administered concurrently or sequentially with respect to administration of the modified poxvirus, such as oncolytic vaccinia virus. In certain embodiments, the methods of this disclosure can comprise administering a modified oncolytic virus as disclosed herein, followed by, preceded by, or in combination with one or more anti-cancer agents or cancer therapies. Anti-cancer agents can include, but are not limited to, chemotherapeutic agents, radiotherapeutic agents, cytokines, immune checkpoint inhibitors, anti-angiogenic agents, apoptosis-inducing agents, anti-cancer antibodies and/or anti-cyclin-dependent kinase agents. In certain embodiments, the cancer therapies can include chemotherapy, biological therapy, radiotherapy, immunotherapy, hormone therapy, anti-vascular therapy, cryotherapy, toxin therapy and/or surgery or combinations thereof. In certain embodiments, the methods of this disclosure can include administering a modified virus, disclosed herein, followed by, preceded by or in combination with an modified oncolytic virus of this disclosure. Combination of the modified oncolytic poxvirus, such as the modified vaccinia virus with chemotherapy achieves a synergistic effect which is not seen in modified oncolytic viruses that do not comprise the modifications in the modified oncolytic virus. The synergistic effect of the above combination can be advantageously used to lower the dose of chemotherapy, such as Taxol®. Thus, the treatment method disclosed here, with the modified virus, can reduced toxicities associated with chemotherapy, e.g., patients who respond to chemotherapy but suffer side effects at therapeutic doses. The synergistic effect, can, in certain cases, result in a decrease in tumor growth compared to chemotherapy alone or oncolytic vaccinia virus alone. Exemplary decrease in tumor growth can be from about 2% to about 50%, such as about 5%, about 10%, about 20%, about 25%, about 35%, about 45% or about 50%.

In certain embodiments, treatment using a modified oncolytic poxvirus, such as a vaccinia virus can be used alone or in combination with one or immunomodulatory agents. An immunomodulatory agent can include any compound, molecule or substance capable of suppressing antiviral immunity associated with a tumor or cancer. In certain embodiments, the immunomodulatory agent can be capable of suppressing innate immunity or adaptive immunity to the modified virus. Non-limiting examples of immunomodulatory agents include anti-CD33 antibody or variable region thereof (also referred to herein as, antigen binding fragments thereof), an anti-CD11b antibody or variable region thereof (also referred to herein as, antigen binding fragments thereof), a COX2 inhibitor, e.g., celecoxib, cytokines, such as IL12. GM-CSF, IL2, IFN3 and IFNγ, and chemokines, such as MIP-1, MCP-1 and IL-8. In certain embodiments, the immunomodulatory agent can include immune checkpoint modulators such as, but not limited to, anti-CTLA4, anti-PD-1, and anti-PD-L1 and TLR agonists Poly I:C), In some examples, the immunomodulatory agent can include an immune checkpoint inhibitor, such as an antagonist of PD-1 (e.g., an antagonist antibody that binds to PD-1), an antagonist of PD-L1 (e.g, an antagonist antibody that binds to PD-L1), an antagonist of CTLA-4 (e.g., an antagonist antibody that binds to CTLA-4), an antagonist of A2AR (e.g., an antagonist antibody that binds to A2AR), an antagonist of B7-H3 (e.g., an antagonist antibody that binds to B7-H3), an antagonist of B7-H4 (e.g., an antagonist antibody that binds to B7-H4), an antagonist of BTLA an antagonist antibody that binds to BTLA), an antagonist of IDO (e.g., an antagonist antibody that binds to IDO), an antagonist of KIR (e.g., an antagonist antibody that binds to KIR), an antagonist of LAG3 (e.g., an antagonist antibody that binds to LAG3), an antagonist of TIM-3 (e.g., an antagonist antibody that binds to TIM3). In some embodiments, the further therapy can comprise administering an immune checkpoint regulator. In one example, the immune checkpoint regulator can be TGN1412. In one example, the immune checkpoint regulator can be NKTR-214. In one example, the immune checkpoint regulator can be MEDI0562. In one example, the immune checkpoint regulator can be MEDI6469. In one example, the immune checkpoint regulator can be MEDI6383. In one example, the immune checkpoint regulator can be TX-2011. In one example, the immune checkpoint regulator can be Keytruda (pembrolizumab). In one example, the immune checkpoint regulator can be Opdivo (nivolumab). In one example, the immune checkpoint regulator can be Yervoy (ipilimumab), In one example, the immune checkpoint regulator can be tremelimumab. In one example, the immune checkpoint regulator can be Tecentriq (atezolizumab). In one example, the immune checkpoint regulator can be MGA271. In one example, the immune checkpoint regulator can be indoximod. In one example, the immune checkpoint regulator can be Epacadostat. In one example, the immune checkpoint regulator can be lirilumab. In one example, the immune checkpoint regulator can be BMS-986016. In one example, the immune checkpoint regulator can be MPDL3280A. In one example, the immune checkpoint regulator can be avelumab. In one example, the immune checkpoint regulator can be durvalumab. In one example, the immune checkpoint regulator can be MEDI4736. In one example, the immune checkpoint regulator can be MEDI4737. In one example, the immune checkpoint regulator can be TRX518. In one example, the immune checkpoint regulator can be MK-4166. In one example, the immune checkpoint regulator can be urelumab (BMS-663513). In one example, the immune checkpoint regulator can be PF-05082566 (PF-2566)

In certain examples, where the further therapy is radiation exemplary doses can be 5,000 Rads (50 Gy) to 100,000 Rads (1000 Gy), or 50,000 Rads (500 Gy), or other appropriate doses within the recited ranges. Alternatively, the radiation dose can be about 30 to 60 Gy, about 40 to about 50 Gy, about 40 to 48 Gy, or about 44 Gy, or other appropriate doses within the recited ranges, with the dose determined, example, by means of a dosimetry study as described above. “Gy” as used herein can refer to a unit for a specific absorbed dose of radiation equal to 100 Rads. Gy is the abbreviation for “Gray.”

In certain examples, where the further therapy is chemotherapy, exemplary chemotherapeutic agents can include without limitation alkylating agents (e.g., nitrogen mustard derivatives, ethylenimines, alkylsulfonates, hydrazines and triazines, nitrosureas, and metal salts), plant alkaloids (e.g., vinca alkaloids, taxanes, podophyllotoxins, and camptothecan analogs), antitumor antibiotics (e.g., anthracyclines, chromomycins, and the like), antimetabolites (e.g., folic acid antagonists, pyrimidine antagonists, purine antagonists, and adenosine deaminase inhibitors), topoisomerase I inhibitors, topoisomerase II inhibitors, and miscellaneous antineoplastics (e.g., ribonucleotide reductase inhibitors, adrenocortical steroid inhibitors, enzymes, antimicrotubule agents, and retinoids). Exemplary chemotherapeutic agents can include, without limitation, anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®), Ibrutinib, idelalisib, and brentuximab vedotin.

Exemplary alkylating agents can include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemantharnine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune®), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®), Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®): Bendamustine (Treanda®); Busulfan (Busulfex® and Myleran®); Carboplatin (Paraplatin®); Lomustine (also known as CCNU, CeeNU®), Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®), Ifosfamide (Ifex®); Prednumustine: Procarbazine (Matulane®), Mechlorelhamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®), and Bendamustine HCl (Treanda®).

Exemplary anthracyclines can include, without limitation, doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunoruhicin citrate liposome, DaunoXome®), mitoxantrone (DHAD, Novainrone®); epirubicin (Ellence™); idarubicin (Idamycin®, Idamycin PFS®); mitomycin C (Mutamycin®); geldanamycin; herbimycin; ravidomycin: and desacetylravidomycin.

Exemplary vinca alkaloids can include, but are not limited to, vinorelbine tartrate (Navelbine®), Vincristine (Oncovin®), and Vindesine (Eldisine®)); vinblastine (also known as vinblastine sulfate, vincaleukoblastine, and VLB, Alkaban-AQ® and Velban®); and vinorelbine (Navelbine®).

Exemplary proteasome inhibitors can, hut are not limited to, bortezomib (Velcade®); carfilzomib (PX-171-007, (S)-4-Methyl-N—((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoac etamido)-4-phenylbutanamido)-pentanamide); marizomib (NPI-0052); ixazomib citrate (MLN-9708), delanzomib (CEP-18770); and O-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912).

“In combination with,” as used herein, means that the modified poxvirus, such as an oncolytic vaccinia virus as described herein or a pharmaceutical composition comprising the same, and the further therapy, such as a further therapy comprising one or more agents are administered to a subject as part of a treatment regimen or plan. In certain embodiments, being used in combination does not require that the modified oncolytic virus and the one or more agents are physically combined prior to administration or that they be administered over the same time frame. For example, and not by way of limitation, the modified oncolytic virus and the one or more agents can be administered concurrently to the subject being treated, or can be administered at the same time or sequentially in any order or at different points in time.

The further therapy can be administered, in various embodiments, in a liquid dosage form, a solid dosage form, a suppository, an inhalable dosage form, an intranasal dosage form, in a liposomal formulation, a dosage form comprising nanoparticles, a dosage form comprising microparticles, a polymeric dosage form, or any combinations thereof. In certain embodiments, the further therapy is administered over a period of about 1 week to about 2 weeks, about 2 weeks to about 3 weeks, about 3 weeks to about 4 weeks, about 4 weeks to about 5 weeks, about 6 weeks to about 7 weeks, about 7 weeks to about 8 weeks, about 8 weeks to about 9 weeks, about 9 weeks to about 10 weeks, about 10 weeks to about 11 weeks, about 11 weeks to about 12 weeks, about 12 weeks to about 24 weeks, about 24 weeks to about 48 weeks, about 48 weeks or about 52 weeks, or longer. The frequency of administration of the further therapy can be, in certain instances, once daily, twice daily, once every week, once every three weeks, once every four weeks (or once a month), once every 8 weeks (or once every 2 months), once every 12 weeks (or once every 3 months), or once every 24 weeks (once every 6 months). In certain embodiments, a method of treating a subject having a cancer can include administering, to the subject, an effective amount of a modified oncolytic poxvirus, such as a modified oncolytic vaccinia virus of this disclosure. In certain embodiments, the methods of this disclosure can further include administering to the subject an effective amount of one or more agents. For example, and not by way of limitation, the agent can be an anti-cancer agent, an immunomodulatory agent, or any combinations thereof, as described above.

### Kits

In embodiments, this disclosure provides for a kit for administering a modified oncolytic poxvirus, such as a modified oncolytic vaccinia virus as described herein. In certain embodiments, a kit of this disclosure can include a modified oncolytic poxvirus, such as a modified oncolytic vaccinia virus or a pharmaceutical composition comprising a modified oncolytic vaccinia virus as described above. In certain embodiments, a kit of this disclosure can further include one or more components such as instructions for use, devices and additional reagents, and components, such as tubes, containers and syringes for performing the methods disclosed above. In certain embodiments, a kit of this disclosure can further include one or more agents, e.g., at least one of an anti-cancer agent, an immunomodulatory agent, or any combinations thereof, that can be administered in combination with a modified virus.

In certain embodiments, a kit of this disclosure can comprise one or more containers containing a modified virus, disclosed herein. For example, and not by way of limitation, a kit of this disclosure can comprise one or more containers that contain a modified oncolytic virus of this disclosure.

In certain embodiments, a kit of this disclosure can include instructions for use, a device for administering the modified oncolytic virus to a subject, or a device for administering an additional agent or compound to a subject. For example, and not by way of limitation, the instructions can include a description of the modified oncolytic virus and, optionally, other components included in the kit, and methods for administration, including methods for determining the proper state of the subject, the proper dosage amount and the proper administration method for administering the modified virus. Instructions can also include guidance for monitoring the subject over duration of the treatment time.

In certain embodiments, a kit of this disclosure can include a device for administering the modified oncolytic virus to a subject. Any of a variety of devices known in the art for administering medications and pharmaceutical compositions can be included in the kits provided herein. For example, and not by way of limitation, such devices include, a hypodermic needle, an intravenous needle, a catheter, a needle-less injection device, an inhaler and a liquid dispenser, such as an eyedropper. In certain embodiments, a modified oncolytic virus to be delivered systemically, for example, by intravenous injection, an intratumoral injection, an intraperitoneal injection, can be included in a kit with a hypodermic needle and syringe.

### EXAMPLES

The examples below further illustrate the described embodiments without limiting the scope of this disclosure.

### Example 1: Preparation and Characterization of a Modified Oncolytic Vaccinia Virus Containing WO34

This study identified and characterized a novel mutation in vaccinia virus (WR strain) that increases release of EEV particles from the virus. The EEV particles can enhance the oncolytic potential of the virus (e.g., through increased delivery and spread through the blood stream), and may provide benefits for other therapeutic uses of vaccinia (e.g., as vaccines).

Random mutagenesis of WR A34R gene was performed and the vaccinia viral strains thus generated (containing various mutations in the A34R gene) were screened for EEV particles production in order to identify strains containing mutations that enhanced production release of high levels of EEV. Strains were identified that contained a K151E mutation along with an additional point mutation in A34R (K119E). It was observed that in case of strains containing the double mutation in A34R (K119E and K151E) (the WO34) there was significant increase in the level of FEY particles production. Additional studies carried out indicated that the WO34 (A34R double mutants K119E and K151E) containing oncolytic strains displayed enhanced therapeutic effects when assessed in the context of cancer treatments in mice models.

### Screening Methods for Identifying WO34

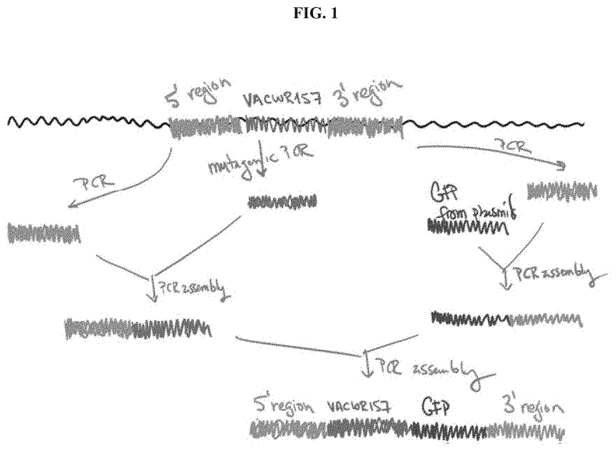

Random mutagenesis of viral gene VACWR157 (also referred to herein as the A34R gene encoding the A34R protein) was carried out using PCR. The A34R open reading frame (ORF) was amplified using Tag polymerase in the presence of nucleotide analogues 8-oxo-dGTP and dPTP. Regions directly 5′ and 3′ of VACWR157 were also amplified by PCR. The latter was assembled with a GFP reporter (GFP on the 5′ end), also by PCR. Finally, the 5′-mutagenized VACWR157, and GFP-3′ fragments were all assembled by PCR, employing short complementary regions between fragments, which were added by PCR primers. A simplified assembly schematic is depicted in FIG. 1 (promoters and primer overhangs are not shown).

The fully assembled fragment containing the mutagenized VACWR157 and GFP flanked by 5′ and 3′ regions of vaccinia virus DNA was purified and used to transfect 143B human osteosarcoma cells. Transfected cells were then infected with vaccinia virus to allow recombination-driven creation of a mutagenized VACWR157 virus library. To preferentially select EEV production-enhanced mutants, medium from flasks infected with the virus pool was added to non-infected flasks. Clonal plaques were isolated after several rounds of serial infection, and mutations in the UV production-enhanced A34R were characterized by Sanger sequencing.

It was observed that all isolated clones shared two missense mutations: K119E and K151E, and one silent mutation at F129 (SEQ ID No. 2). The mutant protein was designated as WO34 (SEQ ID No. 2), and one corresponding clone was assigned UID WO0064R.002. The GFP reporters in this initial cohort of clones were not flanked by loxP sites and cannot be removed. A codon-scrambled ORF encoding WO34 was synthesized and used to clone a transfer vector (pWR157-KE.R) that contained the loxP-flanked GFP. New recombinant viruses that encode WO34 were selected from fluorescent plaques and then used to infect cre recombinase-expressing cells. Reporter-lacking clones were selected after cre treatment.

TABLE 11
A34R gene and protein sequences

| | | |
|---|---|---|
| 1 | A34R | >NC_006998.1:143912-144418 Vaccinia virus, complete |
| | | genome |
| | | ATGAAATCGCTTAATAGACAAACTGTAAGTAGGTTTAAGAAGTTGTCGGTGC |
| | | CGGCCGCTATAATGATGATACTCTCAACCATTATTAGTGGCATAGGAACATT |
| | | TCTGCATTACAAAGAAGAACTGATGCCTAGTGCTTGCGCCAATGGATGGATA |
| | | CAATACGATAAACATTGTTATTTAGATACTAACATTAAAATGTCTACAGATA |
| | | ATGCGGTTTATCAGTGTCGTAAATTACGAGCCAGATTGCCTAGACCGGATAC |
| | | TAGACATCTGAGAGTATTGTTTAGTATTTTTTATAAAGATTATTGGGTAAGT |
| | | TTAAAAAAGACCAATGATAAATGGTTAGATATTAATAATGATAAAGATATAG |
| | | ATATTAGTAAATTAACAAATTTTAAACAACTAAACAGTACGACGGATGCTGA |
| | | AGCGTGTTATATATACAAGTCTGGAAAACTGGTTAAAACAGTATGTAAAAGT |
| | | ACTCAATCTGTACTATGTGTTAAAAAATTCTACAAGTGA |
| 2 | A34R | ATGAAATCGCTTAATAGACAAACTGTAAGTAGGTTTAAGAAGTTGTCGGTGC |
| | modified | CGGCCGCTATAATGATGATACTCTCAACCATTATTAGTGGCATAGGAACATT |
| | | TCTGCATTACAAAGAAGAACTGATGCCTAGTGCTTGCGCCAATGGATGGATA |
| | | CAATACGATAAACATTGTTATTTAGATACTAACATTAAAATGTCTACAGATA |
| | | ATGCGGTTTATCAGTGTCGTAAATTACGAGCCAGATTGCCTAGACCGGATAC |
| | | TAGACATCTGAGAGTATTGTTTAGTATTTTTTATAAAGATTATTGGGTAAGT |
| | | TTAAAAAAGACCAATGATAAATGGTTAGATATTAATAATGATGAGGATATAG |
| | | ATATTAGTAAATTAACAAATTTCAAACAACTAAACAGTACGACGGATGCTGA |
| | | AGCGTGTTATATATACAAGTCTGGAAAACTGGTTGAAACAGTATGTAAAAGT |
| | | ACTCAATCTGTACTATGTGTTAAAAAATTCTACAAGTGA |
| 3 | A34R | atgaaatcgcttaatagacaaactgtaagtaggtttaagaagttgtcggtgc |
| | mutated | cggccgctataatgatgatactctcaaccattattagtggcataggaacatt |
| | (nucleic | tctgcattacaaagaagaactgatgcctagtgcttgcgccaatggatggata |
| | acid | caatacgataaacattgttatttagatactaacattaaaatgtctacagata |
| | sequences) | atgcggtttatcagtgtcgtaaattacgagccagattgcctagaccggatac |
| | (nucleic | tagacatctgagagtattgtttagtattttttataaagattattaggtaagt |
| | acid | ttaaaaaagaccaatgataaatagttagatattaataatgatGaGgatatag |
| | differences | atattagtaaattaacaaatttCaaacaactaaacagtacgacggatgctga |
| | with wild | agcgtgttatatatacaagtctggaaaactggttGaaacagtatgtaaaagt |
| | type A34R | actcaatctgtactatgtgttaaaaaattctacaagtga |
| | coding | |
| | sequence | |
| | are bold, | |
| | and mutated | |
| | codons | |
| | underlined | |
| 4 | Wild-type | >sp\|P24761\| A34_VACCW Protein A34 OS = Vaccinia virus |
| | A34R | (strain Western Reserve) OX = 10254 GN = VACWR157 PE = 1 |
| | protein | SV = 1 |
| | | MKSLNRQTVSRFKKLSVPAAIMMILSTIISGIGTFLHYKEELMPSACANGWI |
| | | QYDKHCYLDTNIKMSTDNAVYQCRKLRARLPRPDTRHLRVLFSIFYKDYWVS |
| | | LKKTNDKWLDINNDKDIDISKLTNFKQLNSTTDAEACYIYKSGKLVKTVCKS |
| | | TQSVLCVKKFYK |
| 5 | WO34 (A34 | MKSLNRQTVSRFKKLSVPAAIMMILSTIISGIGTFLHYKEELMPSACANGWI |
| | double mutant | QYDKHCYLDTNIKMSTDNAVYQCRKLRARLPRPDTRHLRVLFSIFYKDYWVS |
| | K119E | LKKTNDKWLDINNDEDIDISKLTNFKQLNSTTDAEACYIYKSGKLVETVCKS |
| | K151E) | TQSVLCVKKFYK* |

### Viral Plaque Comet Tails Firmed by EEV-Enhanced Vaccinia Virus Containing WO34

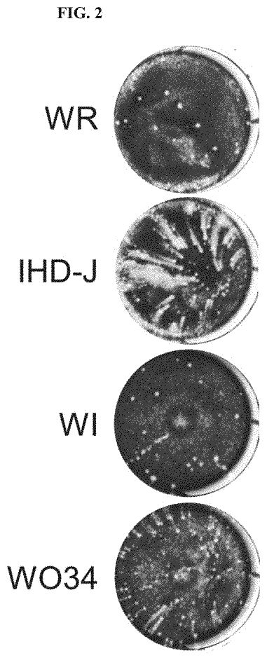

EEV-enhanced vaccinia viruses that were previously titered were diluted and placed over confluent BS-C-40 cells in 6-well plates. Plates were incubated and left undisturbed for 48 hours and then the cells were stained with crystal violet to view differences in comet tail formation caused by aberrant EEV production. Results are shown in FIG. 2. As is seen in FIG. 2, the WO34 containing vaccinia virus strain demonstrated enhanced comet-tail formation compared to the WI strain (A34R single mutation in WR strain) and WR strain (non-mutated A34R). The comet-tail formation of the WO34 containing vaccinia virus strain was visibly increased.

### Neutralization of the EEV-Enhanced Vaccinia Virus Containing WO34

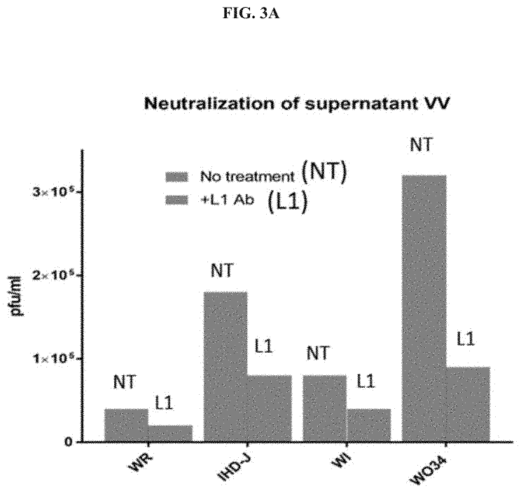

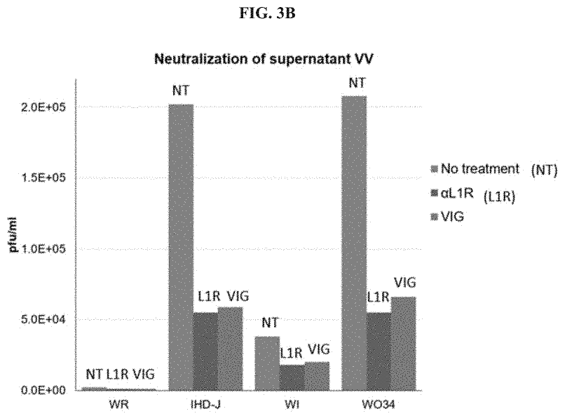

in two separate experiments using different strains of vaccinia viruses (WO34 containing vaccinia virus, WI strain, IHD-J strain, and WR strain), HeLa cells were grown to confluency in 6-well plates and infected with 1 MOI (multiplicity of infection) of a vaccinia virus strain. After 24 hours, 1 mL of culture medium was removed and centrifuged at 800 g. About 500 μl of supernatant was then removed and used for viral plaque assay. During the serial dilution, samples were treated with anti-L1 NR-45114 antibody or VIG then incubated for 1 hour at 37° C. Dilutions were then added to confluent 6 well plates of BS-C-40 cells for plaque assay. After 1.5 hours, media was replaced with CM10 with 3% CMC. After 48 hours, cells were stained with crystal violet to count viral plaques to determine the titer of virus in the HeLa cell supernatant and the blocking ability of neutralizing antibodies as shown in FIGS. 3A-3B (FIG. 3A shows viral plaque titer followed by treatment with the anti-L1 NR-45114 antibody and FIG. 3B shows the same followed by treatment with the anti-L1R antibody and the VIG antibody).

Cell Viability after Infection with EEV-Enhanced Vaccinia Virus Containing WO34

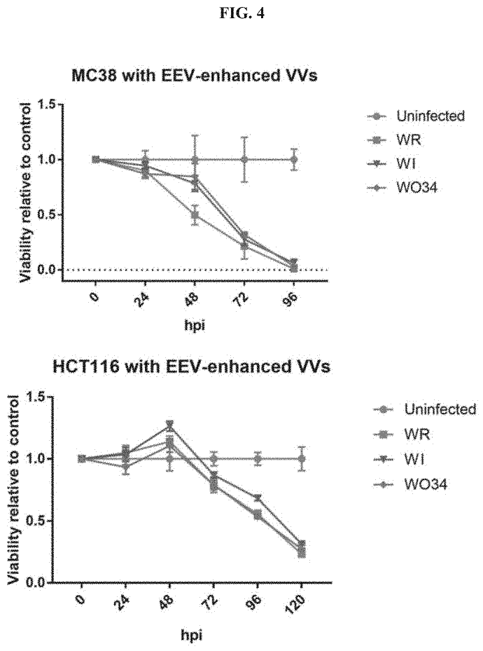

In 96-well plates, HCT116 or MC38 cell lines were seeded and allowed to grow until 90% confluent. Cells were then infected with 1 MOI of different vaccinia virus strains (WR strain, IHD-J strain, WI strain, WO34 containing strain). Each day, at 24 hour intervals, cell viability was tested with the CellTiter 96 Aqueous Non-radioactive Cell Proliferation Kit from Promega. Relative viability was calculated by removing the blank value average from all wells, calculating the average value of the uninfected control group, then calculating the relative value of each infected well as (A490/average of uninfected wells). Results are shown in FIG. 4 (upper panel-MC38 cells; lower panel HCT116 cells).

### Viral Replication Assay of EEV-Enhanced Vaccinia Virus Containing WO34 in Cancer Cells

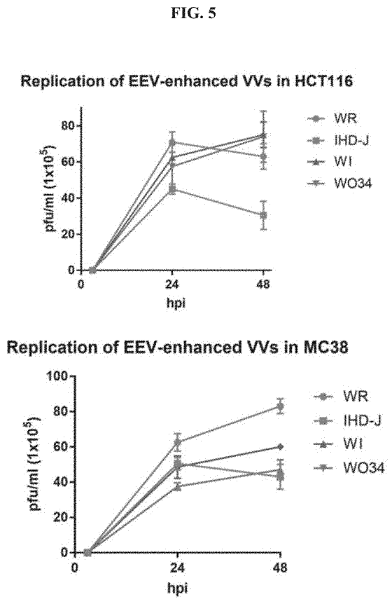

In a separate experiment, HCT116 or MC38 cells were grown on 12-well plates to 90% confluency, with 1 plate used for each day of a 3 day replication assay. All plates were infected on the same day with 1 MOT of different vaccinia virus strains (WR, strain, IHD-J strain, WI strain, WO34 containing strain), For every 2411 post-infection, 1 plate was frozen in a −80° C. freezer. Plates were frozen and thawed 2 times to break cells and the lysate was used for a viral plaque assay on BS-C-40 cells. Plaque forming units per ml are shown in FIG. 5 (upper panel HCT116 cells; lower panel MC38 cells).

1. A modified oncolytic poxvirus, wherein the modified oncolytic virus comprises:
a nucleic acid that codes for an A34R protein or a fragment thereof, wherein the nucleic acid comprises at least two mutations that each incorporate an amino acid residue negatively charged at pH 5, and wherein the A34R protein is mutated relative to a sequence within a wild-type vaccinia virus A34R protein (SEQ ID No, 4).

2. The modified oncolytic poxvirus of claim 1, wherein the at least two mutations are in positions corresponding to positions Lys119 and Lys151 of the wild-type vaccinia virus A34R protein (SEQ ID NO. 4).

3. The modified oncolytic poxvirus of claim 2, wherein the mutation in the position corresponding to position Lys119 is Lys119Glu.

4. The modified oncolytic poxvirus of claim 2, wherein the mutation in the position corresponding to position Lys151 is Lys151Glu.

5. The modified oncolytic poxvirus of claim 2, wherein the at least two mutations in positions corresponding to positions Lys119 and Lys151 of the wild-type vaccinia virus A34R protein (SEQ ID NO. 4) are Lys119Glu and Lys151Glu, respectively.

6. The modified oncolytic poxvirus of claim 1, wherein the mutations are not at position 110 of the wild-type A34R protein (SEQ ID No. 4).

7. The modified oncolytic poxvirus of claim 1, wherein the mutations are not at aspartic acid residues within the wild-type A34R protein (SEQ ID No. 4).

8. The modified oncolytic poxvirus of claim 1, wherein one of the mutations is at position Lys1119 of the wild-type A34R protein (SEQ ID No. 4).

9. The modified oncolytic poxvirus of claim 1, wherein the modified oncolytic poxvirus generates an increased number of comet tail type plaques in a viral plaque forming assay, compared to an otherwise identical oncolytic poxvirus that does not comprise the at least two mutations.

10. The modified oncolytic poxvirus of claim 1, wherein the poxvirus is a vaccinia virus.

11. A modified oncolytic poxvirus that expresses an A34R protein comprising mutations Lys119Glu and Lys1.51Glu.

12. The modified oncolytic poxvirus of claim 11, wherein the modified oncolytic poxvirus produces a greater amount of an extracellular enveloped virus form than an intracellular mature virus form, as compared to an otherwise identical oncolytic virus that does not comprise the at least two mutations Lys119Glu and Lys151Glu.

13. The modified oncolytic poxvirus of claim 11, further comprising the mutation or deletion of viral gene A52R.

14. A method of treating a tumor, the method comprising administering to a subject a composition comprising patient-derived leukocyte cells infected with a modified oncolytic poxvirus that expresses an A34R protein comprising mutations at positions 119 and 151 of a wild-type A34 protein (SEQ ID No. 4), wherein the modified oncolytic poxvirus produces a population of viral particles in a tumor microenvironment.

15. The method of claim 14, wherein the patient-derived leukocyte cells comprise macrophages.

16. The method of claim 14, wherein the patient-derived leukocyte cells comprise tumor-targeted T cells.

17. The method of claim 14, wherein at least about 10% to at least about 90% of the population of viral particles are EEV particles, as measured in a viral plaque assay.

18. The method of claim 17, further comprising harvesting the EEV particles from the tumor microenvironment and intravenously administering the EEV particles to the subject.

19. The method of claim 14, wherein the administering is via an intratumoral injection, an intravenous injection, or a combination thereof.

20. The method of claim 19, wherein the method further comprises administering a further therapy, in combination with the oncolytic poxvirus, wherein the further therapy comprises at least one of: a chemotherapy, a radiation therapy, an oncolytic viral therapy with an additional virus, treatment with an immunomodulatory protein, a CAR T cellular therapy, an anti-cancer agent, an immunomodulatory agent, or any combinations thereof.


SEQUENCE LISTING (5 sequences)

SEQ ID NO: 1 - DNA, 507 bp, Vaccinia virus
atgaaatcgcttaatagacaaactgtaagtaggtttaagaagttgtcggtgccggccgctataatgatgatactctcaaccattattagtggcataggaacatttctgcattacaaagaagaactgatgcctagtgcttgcgccaatggatggatacaatacgataaacattgttatttagatactaacattaaaatgtctacagataatgcggtttatcagtgtcgtaaattacgagccagattgcctagaccggatactagacatctgagagtattgtttagtattttttataaagattattgggtaagtttaaaaaagaccaatgataaatggttagatattaataatgataaagatatagatattagtaaattaacaaattttaaacaactaaacagtacgacggatgctgaagcgtgttatatatacaagtctggaaaactggttaaaacagtatgtaaaagtactcaatctgtactatgtgttaaaaaattctacaagtga

SEQ ID NO: 2 - DNA, 507 bp, Artificial Sequence
  Description of Artificial Sequence Synthetic polynucleotide
atgaaatcgcttaatagacaaactgtaagtaggtttaagaagttgtcggtgccggccgctataatgatgatactctcaaccattattagtggcataggaacatttctgcattacaaagaagaactgatgcctagtgcttgcgccaatggatggatacaatacgataaacattgttatttagatactaacattaaaatgtctacagataatgcggtttatcagtgtcgtaaattacgagccagattgcctagaccggatactagacatctgagagtattgtttagtattttttataaagattattgggtaagtttaaaaaagaccaatgataaatggttagatattaataatgatgaggatatagatattagtaaattaacaaatttcaaacaactaaacagtacgacggatgctgaagcgtgttatatatacaagtctggaaaactggttgaaacagtatgtaaaagtactcaatctgtactatgtgttaaaaaattctacaagtga

SEQ ID NO: 3 - DNA, 507 bp, Artificial Sequence; identical to SEQ ID NO: 2
  Description of Artificial Sequence Synthetic polynucleotide

SEQ ID NO: 4 - protein, 168 aa, Vaccinia virus
MKSLNRQTVSRFKKLSVPAAIMMILSTIISGIGTFLHYKEELMPSACANGWIQYDKHCYLDTNIKMSTDNAVYQCRKLRARLPRPDTRHLRVLFSIFYKDYWVSLKKTNDKWLDINNDKDIDISKLTNFKQLNSTTDAEACYIYKSGKLVKTVCKSTQSVLCVKKFYK

SEQ ID NO: 5 - protein, 168 aa, Artificial Sequence
  Description of Artificial Sequence Synthetic polypeptide
MKSLNRQTVSRFKKLSVPAAIMMILSTIISGIGTFLHYKEELMPSACANGWIQYDKHCYLDTNIKMSTDNAVYQCRKLRARLPRPDTRHLRVLFSIFYKDYWVSLKKTNDKWLDINNDEDIDISKLTNFKQLNSTTDAEACYIYKSGKLVETVCKSTQSVLCVKKFYK